(19) United States
(12) Patent Application Publication
(10) Pub. No.: US 2011/0098238 A1
(43) Pub. Date: Apr. 28, 2011

(54) ELUCIDATING LIGAND-BINDING INFORMATION BASED ON PROTEIN TEMPLATES

(75) Inventors: Jeffrey Skolnick, Roswell, GA (US); Michal Brylinski, Atlanta, GA (US)

(73) Assignee: GEORGIA TECH RESEARCH CORPORATION, Atlanta, GA (US)

(21) Appl. No.: 12/809,020

(22) PCT Filed: Dec. 22, 2008
(86) PCT No.: PCT/US08/88071

§ 371 Date: Jul. 19, 2010

Related U.S. Application Data
(60) Provisional application No. 61/015,271, filed on Dec. 20, 2007.

(51) Int. Cl. A61K 31/7034; C40B 30/02; C40B 60/12; A61K 31/661; A61K 31/7076; A61K 31/7072; A61K 31/7056; A61K 31/445; A61K 31/46; A61K 31/437; A61K 31/047; A61K 31/7004; A61K 31/191; A61K 31/7016; A61P 31/18

(52) U.S. Cl. 514/24; 506/8; 506/39; 514/119; 514/48; 514/51; 514/52; 514/315; 514/304; 514/299; 514/738; 514/23; 514/557; 514/25; 514/53; 514/129

(57) ABSTRACT

A method, computer-readable medium, and system for identifying compounds from chemical libraries that can be used for the therapeutic treatment of a disease or used as lead compounds in a drug development program. In particular, information from homologous proteins is used to predict, for a target protein, molecular functions that can be used to screen libraries of compounds for individual compounds that are predicted to have high binding affinities for the target protein.

### CLAIM OF PRIORITY

This application claims the benefit of priority under 35 U.S.C. §119(e) to U.S. provisional application Ser. No. 61/015,271 filed Dec. 20, 2007, the contents of which are incorporated herein by reference in their entirety.

### STATEMENT OF FEDERALLY SPONSORED RESEARCH OR DEVELOPMENT

The U.S. Government may have certain rights in this invention pursuant to Grant No. GM-48835 awarded by the National Institutes of Health (NIH).

### TECHNICAL FIELD

The technology described herein relates to methods for identifying compounds from chemical libraries that can be used for the therapeutic treatment of a disease or used as lead compounds in a drug development program. In particular, information from homologous proteins is used to predict, for a target protein, molecular functions that can be used to screen libraries of compounds for individual compounds that are predicted to have high binding affinities for the target protein.

### BACKGROUND

Proteins' functional sites play an essential role in cell biology, enabling proteins to interact with ligands. The function of many proteins involved in signal transduction, catalysis and transport can be efficiently modulated by specific ligands; therefore binding sites are the primary targets in small-molecule lead discovery. Genome sequencing projects have generated a huge amount of sequence information. However, the biological function of many identified genes and gene products still remain unknown. In the post-genomic era, the detection of functional sites is typically the starting point for protein function identification followed by drug development and discovery.

Drug design and development is a tremendously time consuming and expensive process. High-throughput screening (HTS) assays have been developed to test a large number of diverse chemical structures against disease targets to identify new biopharmaceuticals. Virtual screening techniques can be used to reduce the costs of experiments and increase the success rate by limiting the size of a screening library to the compounds that are the most likely to display the desired biological activity. Receptor-based virtual screening matches a collection of small molecules against the three-dimensional structure of a target protein by molecular docking of individual compounds into the protein's active-site, and further ranking of the compounds according to the calculated interaction energies and estimated binding affinities.

Ligand-based techniques prioritize the lead molecules based on the molecular similarity to known active compounds. One of the most commonly used computational techniques for high-throughput ligand-based virtual screening is a similarity search that employs molecular fingerprints. Molecular fingerprints consist of linear bit strings encoding chemical and structural properties of organic compounds that can be easily compared using a variety of similarity metrics. Virtual screening experiments using molecular fingerprints can require ligand templates used as query compounds. Typically, for a given target protein, already known active compounds can be used as multiple query compounds. Furthermore, when many active compounds known to exhibit a particular biological activity are available, class-specific profiles can be constructed from characteristic patterns of bits to increase the performance of similarity search calculations. Conventional ligand-based techniques cannot be applied to proteins that have not been yet annotated and their exact molecular function remains unknown due to the lack of information concerning molecular properties of potential ligands.

### SUMMARY

A method for identifying a binding site of a target protein, the method comprising: obtaining a set of protein structure templates for the target protein; selecting a subset of the protein structure templates such that, for each template in the subset, at least one bound ligand conformation is available; optimizing a structural alignment of one or more templates in the subset to a structure of the target protein; calculating a center of mass of each bound ligand conformation in the optimized structural alignment; clustering the centers of mass of the bound ligand conformations in the optimized structural alignment, thereby identifying locations of one or more binding sites of the target protein.

A method of ranking two or more binding sites of a target protein, the method comprising: identifying locations of the two or more binding sites of the target protein by the method as described elsewhere herein; and assigning a rank to each of the two or more binding sites according to a number of bound ligand conformations clustered together at the location of each binding site.

A method of annotating a target protein with at least one molecular function, the method comprising: ranking two or more binding sites of the target protein according to methods as described elsewhere herein; for the highest ranked binding site: retrieving, from a gene ontology database, gene ontology terms for one or more of the templates whose bound ligand conformations are clustered together in the binding site; and annotating the target protein with the gene ontology terms.

A method of screening a target protein for ligands that bind to a binding site on the target protein, the method comprising: identifying a location of the binding site of the target protein by methods as described elsewhere herein; clustering the ligands in the optimized structural alignment of the bound ligand conformations for the binding site; choosing a representative ligand from the cluster; and comparing the representative ligand with a database of ligands.

A method of treating an individual suffering from a disease mediated by a target protein, the method comprising: administering to the individual a ligand identified as binding to the target protein by methods as described elsewhere herein, in an amount sufficient to produce a therapeutic effect.

The present technology further comprises computer systems configured to carry out the methods described herein in whole or in part, and to provide results of said methods to a user, as for example on a display or in the form of a printout.

The present technology further comprises computer-readable media, encoded with computer-executable instructions for carrying out the methods described herein in whole or in part, when operated on by a suitably configured computer.

When it is stated that a computer system is configured to carry out a method in whole or in part, or that a computer readable medium is configured with instructions for carrying out a method in whole or in part, it is understood to mean that one or more steps of the method is carried out, other than by the computer or computer system. For example, obtaining gene expression data may be obtained manually and read into the computer, or written on to a computer-readable medium.

The details of one or more embodiments of the invention are set forth in the accompanying drawings and the description herein. Other features, objects, and advantages of the invention will be apparent from the description and drawings, and from the claims.

### BRIEF DESCRIPTION OF THE DRAWINGS

FIG. 1 is a flow chart depicting a high-level method for a macromolecular structural template-based method used to elucidate ligand-binding information about a target protein, in accordance with some embodiments.

FIG. 2 is a graphical representation of a high-level method for a macromolecular structural template-based method used to elucidate ligand-binding information about a target protein, in accordance with some embodiments.

FIG. 3 is a flow chart depicting a method for determining one or more structural templates for use with a target protein and subsequently aligning the structural templates to a predicted structure of the target protein, in accordance with some embodiments.

FIG. 4 is a flow chart depicting a method for predicting putative binding sites of a target protein and characteristics of a ligand predicted to bind with a high affinity to the target protein, in accordance with some embodiments.

FIG. 5A depicts structural templates including bound ligands superimposed onto a target protein, where the template proteins are not shown, in accordance with some embodiments.

FIG. 5B depicts the target protein and superimposed structural templates of FIG. 5A, with only the bounds ligands shown.

FIG. 6 is a flow chart depicting a method for detecting the anchor residues in contact with a ligand that are strongly conserved over the course of evolution that are crucial for ligand binding.

FIG. 7 is a flow chart depicting a method for identifying compounds, from one or more chemical compound libraries, that are predicted to bind to a target protein, in accordance with some embodiments.

FIG. 8 depicts an exemplary computer system that can perform the methods described herein, in accordance with some embodiments.

FIG. 9 depicts the average molecule size±SD (one standard deviation) plotted as the function of average anchor size for the largest clusters of similar compounds bound to the top-ranked predicted binding pockets, in accordance with exemplary embodiments.

FIG. 10 depicts the average sequence entropy and normalized B-factor for the Cα atoms calculated for the anchor and non-anchor CBRs, in accordance with exemplary embodiments.

FIG. 11A depicts the average normalized B-factor for side chain heavy atoms, in accordance with exemplary embodiments.

FIG. 11B depicts a random property assigned to anchor and non-anchor CBRs., in accordance with exemplary embodiments.

FIG. 12 depicts the cumulative distribution of the average pairwise RMSD of the anchor substructures upon global superposition of template proteins, in accordance with exemplary embodiments.

FIG. 13A depicts the ligand pose (thick, solid) of a superimposed ligand determined by FINDSITE^{LHM} for the human fibroblast collagenase (PDB-ID: 1hfc) and the experimental binding pose (thin, transparent), in accordance with exemplary embodiments.

FIG. 13B depicts the ligand pose (thick, solid) and the experimental binding pose (thin, transparent) of FIG. 13A, after energy minimization.

FIG. 13C depicts the ligand pose (thick, solid) generated by AutoDock for the human fibroblast collagenase (PDB-ID: 1hfc) and the experimental binding pose (thin, transparent), in accordance with exemplary embodiments.

FIGS. 14A-C depict the accuracy of ligand docking by FINDSITE^{LHM}, in accordance with exemplary embodiments.

FIG. 15 depicts the coverage of conserved enzyme substrate substructures by anchor and non-anchor functional groups from 35 ligand clusters identified for 24 enzymes, in accordance with exemplary embodiments.

FIG. 16A depicts the sequence entropy (light gray—low, light gray—high) for the binding site of glutathione sulfonic acid complexed with glutathione S-transferase (PDB-ID: 1a0f), in accordance with exemplary embodiments.

FIG. 16B depicts the normalized crystallographic B-factors (light gray—low, light gray—high) for the binding site of the complex of FIG. 16A.

FIG. 16C depicts random values (light gray—0.0, light gray—1.0) for the binding site of the complex of FIG. 16A.

FIG. 17A depicts the sequence entropy (light gray—low, light gray—high) for the binding site of 5′-methylthiotubercidin complexed with MTA phosphorylase (PDB-ID: 1sd2), in accordance with exemplary embodiments.

FIG. 17B depicts the normalized crystallographic B-factors (light gray—low, light gray—high) for the binding site of the complex of FIG. 17A.

FIG. 17C depicts random values (light gray—0.0, light gray—1.0) for the binding site of the complex of FIG. 17A.

FIG. 18A depicts the sequence entropy (light gray—low, light gray—high) for the binding site of lysine and piridoxal-5′-phosphate complexed with lysine aminotransferase (PDB-ID: 2cjd), in accordance with exemplary embodiments.

FIG. 18B depicts the normalized crystallographic B-factors (light gray—low, light gray—high) for the binding site of the complex of FIG. 18A.

FIG. 18C depicts random values (light gray—0.0, light gray—1.0) for the binding site of the complex of FIG. 18A.

FIG. 19 depicts ligand poses (thick sticks) for glutathione S-transferase (PDB-ID: 1a0f) from (A) FINDSITE^{LHM} superimposed ligand, (B) FINDSITE^{LHM} superimposed ligand minimized with Amber; (C) AutoDock; (D) LIGIN; (E) Q-Dock and (F) a randomly placed ligand with respect to the experimental binding pose (thin sticks), in accordance with some embodiments.

FIG. 20 depicts ligand poses (thick sticks) for MTA phosphorylase (PDB-ID: 1sd2) from (A) FINDSITE^{LHM} superimposed ligand, (B) FINDSITE^{LHM} superimposed ligand minimized with Amber; (C) AutoDock; (D) LIGIN; (E) Q-Dock and (F) a randomly placed ligand with respect to the experimental binding pose (thin sticks), in accordance with some embodiments.

FIG. 21 depicts ligand poses (thick sticks) for lysine aminotransferase (PDB-ID: 2cjd) from (A) FINDSITE^{LHM} superimposed ligand, (B) FINDSITE^{LHM} superimposed ligand minimized with Amber; (C) AutoDock; (D) LIGIN; (E) Q-Dock and (F) a randomly placed ligand with respect to the experimental binding pose (thin sticks), in accordance with some embodiments.

FIG. 22 depicts the enrichment behavior for FINDSITE (molecular fingerprints) and FINDSITE^{LHM} (anchor coverage) approaches compared to a random ligand selection in virtual screening for HIV-1 protease inhibitors, in accordance with some embodiments.

Like reference symbols in the various drawings indicate like elements.

### DETAILED DESCRIPTION

In some embodiments, a macromolecular structure template-based system, such as a computer-based system, that utilizes various data such as structural information of weakly homologous proteins, can be used to elucidate ligand-binding information about a target protein belonging to a species (e.g., human). The system described here can use a combination of information regarding a target protein whose three dimensional structure may or may not be known and information from structural templates to make predictions about the molecular function of the target protein (e.g., specific binding of ligands to the target protein, amino acid residues involved in ligand binding, or the like). Information determined about molecular function can be further used, for example, to screen libraries of chemical compounds to rank compounds included in the library in order of predicted binding affinity. Furthermore, due to the speed and automated nature of the system, compounds predicted to have a high binding affinity for a target protein could optionally be screened against the entirety of known proteins associated with a species for the purpose of identifying compounds that have low binding affinities for other proteins belonging to the species. In this way, compounds can be identified that have a high binding affinity for the target protein (e.g., maximizing a desired effect) and have low binding affinities for the remaining proteins associated with the species (e.g., minimizing side effects).

For example, HIV-1 protease has been identified as playing an important role in the life cycle of HIV. For this reason, the identification of compounds that have a high affinity for HIV-1 protease can be useful in finding compounds that inhibit the function of HIV-1 protease by, for example, competitively binding to the active site, thus blocking the cleavage of viral polyproteins. In this example, the template-based system can be used to identify one or more ligand templates that are indicative of ligands that have a high affinity for HIV-1 protease. The ligand templates determined by the template-based system can be compared to compound databases, such as the MDL Data Drug Report (http://www.mdl.com/), Asinex Platinum Collection of lead-like compounds (http://www.asinex.com/), or the like for the purpose of identifying compounds that are predicted to have a high binding affinity for HIV-1 protease. In another example, the template-based system can be used to predict molecular function information about HIV-1 protease, such as amino acid residues that are involved in ligand binding. This information can be used to virtually dock compounds to HIV-1 protease for the purpose of identifying compounds that have a high binding affinity for HIV-1 protease and ranking those compounds in order of binding affinity.

When libraries of compounds are analyzed by the template-based system in an automated fashion, some of the compounds predicted by the system to have a high binding affinity for a target protein will have effects that are previously known. These known compounds can serve as relative indicators of the efficacy of the other predicted compounds. For example, compounds that are predicted to a have a higher binding affinity than compounds that are known inhibitors may be predicted to also be inhibitors and thus good candidates for further study.

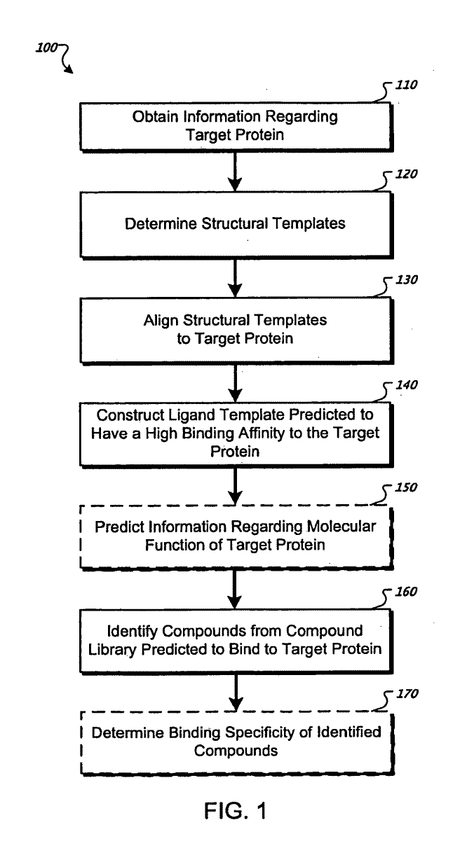

Referring now to FIG. 1, a process 100 for elucidating ligand-binding information can be included in a computational method, such as encoded on a computer-readable medium, in whole or in part, and performed on a computer, in whole or in part. In some embodiments, the process 100 can execute operation 110, causing the template-based system to obtain a target amino acid sequence (e.g., the amino acid sequence of a target protein, a portion of a target protein, or the like) to which ligand binding information is desired.

In some embodiments, the process 100 can execute operation 120, causing the template-based system to determine protein-containing templates, from a larger set of protein information, for use in elucidating ligand-binding information regarding the target protein. For example, the template-based system can parse a non-redundant protein library, such as the Protein Data Bank (PDB), and identify individual protein-containing sequences that have at least some similarity (e.g., homologous, contain one or more similar folds, or the like) to the target protein and include a bound ligand. These sequences, and associated structural information, can be used as a template. During operation 130, the template-based system can align the individual structural templates to the predicted or known experimental structure of the target protein using structural alignment algorithms such as those used by TM-Align (Zhang, Y. and J. Skolnick, TM-align: a protein structure alignment algorithm based on the TM-score. Nucleic Acids Res, 2005. 33(7): p. 2302-9.). The resulting superimposed proteins can be used to determine similarity, superimpose ligands found in the templates onto the target protein, or the like.

In operation 140, the template-based system can construct ligand templates that include partial ligand substructures, which would be predicted to have a high binding affinity for the target protein. For example, using information obtained from the structural alignments performed during operation 130, the template-based system can predict characteristics of ligands that would increase the binding affinity of a ligand for the target protein. The system can use these characteristics (e.g., atoms, functional groups, their positions, or the like) to construct templates of ligands that include partial ligand substructures, which would be predicted to have a binding affinity for the template protein.

In some embodiments, during operation 150, the template-based system can use the templates to predict aspects of the molecular function of the target protein. For example, the system can identify and rank putative binding sites (e.g., by modeling the target protein, structurally aligning the target protein, or the like). The templates determined in operation 120 can include structural information (e.g., as determined from crystallography, NMR data, or the like) about proteins and ligands that are bound to them. Similarities between the target protein and these templates can be used to not only model the target protein (e.g., using homology modeling, protein threading, or the like), but also predict information about binding sites on the target protein (e.g., by superimposing one or more of the ligand-bound templates onto the target protein structure, which can be predicted or experimentally determined). Information determined in operation 140 can also be used to rank the binding sites in order of confidence. In another example, the template-based system can identify consensus binding residues (CBRs) and anchor residues within the protein. CBRs are amino acid residues in the target protein that are predicted to be involved in ligand binding, and anchor residues are those CBRs that are conserved across multiple structural templates. For example, amino acid residues that are shown to be involved in binding in multiple templates can be used to predict amino acid residues in the target protein that may be involved in ligand binding.

In operation 160, the template-based system can identify compounds from a compound library that are predicted to bind with a high affinity to the target protein. For example, the system can parse large compound libraries (e.g., a library with one million compounds) and identify a subset (e.g., one thousand compounds) of compounds that are most likely to bind to the target protein. In operation 170, the subset of compounds can be further analyzed using other techniques to identify one or more compounds with the greatest affinity for the target protein. In another example, ligands from a library can be virtually docked to a subset of the amino acid residues belonging to the target protein, such as the anchor residues described in connection with operation 150.

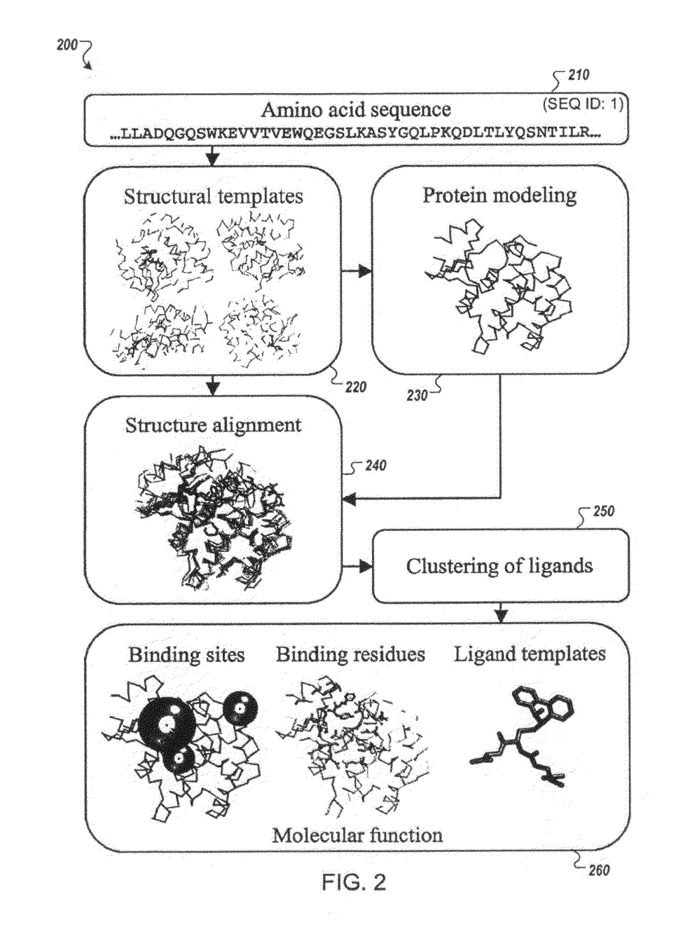

Referring now to FIG. 2, a process 200 for elucidating ligand-binding information can be included in a computational method, such as encoded on a computer-readable medium, in whole or in part, and performed on a computer, in whole or in part. In some embodiments, the process 200 can execute operation 210, causing the template-based system to obtain a target amino acid sequence (e.g., the amino acid sequence of a target protein, a portion of a target protein, or the like) to which ligand binding information is desired. During operation 220, the template-based system can determine structural templates, from a larger set of protein information, for use in elucidating ligand-binding information regarding the target protein. For example, the template-based system can use software such as PROSPECTOR_{—}3 (Skolnick, J., D. Kihara, and Y. Zhang, Development and large scale benchmark testing of the PROSPECTOR_{—}3 threading algorithm. Proteins, 2004. 56(3): p. 502-18.) to parse the non-redundant PDB library and identify individual protein-containing sequences that have at least some similarity (e.g., homologous, contain one or more similar folds, or the like) to the target protein and include a bound ligand. These sequences, and associated structural information, can be used as a template to model the target protein. In operation 230, if the target protein structure is not known, the template-based system can use the protein-containing templates to create a structural model of the amino acid sequence. Software such as PROSPECTOR_{—}3 (Skolnick, J., D. Kihara, and Y. Zhang, Development and large scale benchmark testing of the PROSPECTOR_{—}3 threading algorithm. Proteins, 2004. 56(3): p. 502-18.), MODELLER (Sali, A. and T. L. Blundell, Comparative protein modelling by satisfaction of spatial restraints. J Mol Biol, 1993. 234(3): p. 779-815.), TASSER (Zhang, Y., A. K. Arakaki, and J. Skolnick, TASSER: an automated method for the prediction of protein tertiary structures in CASP6. Proteins, 2005. 61 Suppl 7: p. 91-8.), SWISS-MODEL (Schwede, T., et al., SWISS-MODEL: An automated protein homology-modeling server. Nucleic Acids Res, 2003. 31(13): p. 3381-5.), or the like can be used to predict the structure of the target protein from the determined templates. In operation 240, multiple structural alignments can be performed, by software such as TM-Align (Zhang, Y. and J. Skolnick, TM-align: a protein structure alignment algorithm based on the TM-score. Nucleic Acids Res, 2005. 33(7): p. 2302-9.), where each alignment superimposes one of the templates, determined in operation 220, including any bound ligands, onto the target protein structure determined in operation 230.

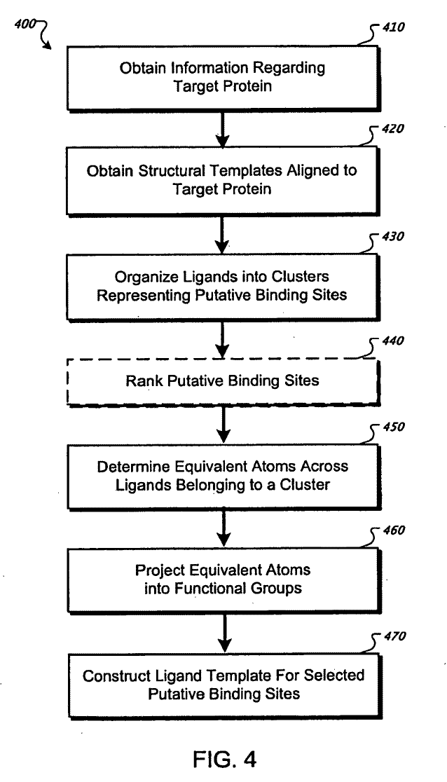

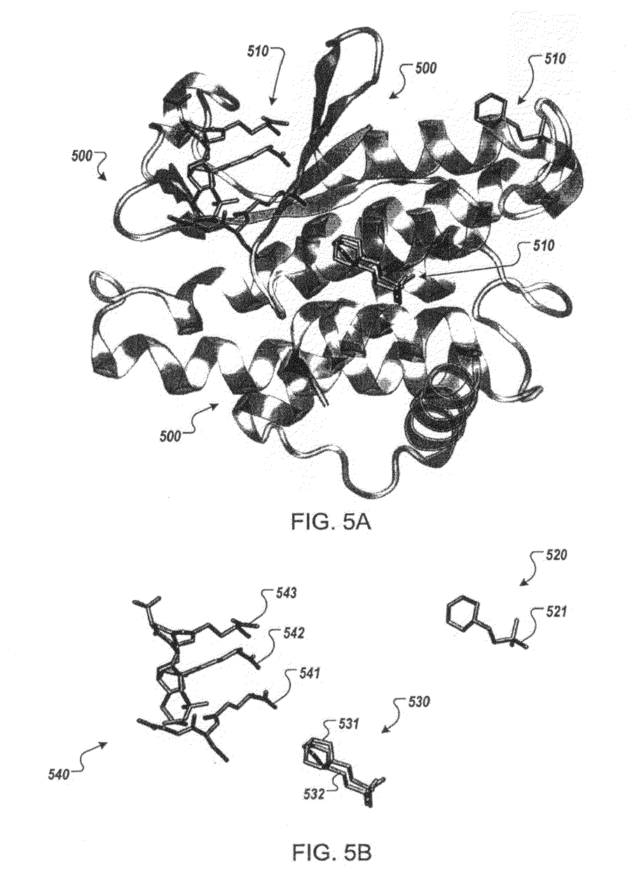

In some embodiments, the process 200 can execute operation 250, causing the template-based system to perform ligand clustering. For example, when superimposing the templates onto the target protein (e.g., as in operation 240), ligands included in the templates are also superimposed onto the target protein. When superimposed, some ligands may be located in close proximity to each other, while other ligands may be found in different locations around the protein. To identify putative binding sites, the system can “cluster” the ligands using a cut-off RMSD (root mean squared deviation) value (e.g., 8 Å). In this example, ligands that fall within 8 Å RMSD of each other are considered part of a single cluster. Each individual ligand cluster can be used later to predict a putative binding site. Ligand clustering is further described in connection with FIGS. 4, 5A, and 5B.

Still referring to FIG. 2, in operation 260, the template-based system can use the superimposed proteins and ligands to elucidate information about the molecular function of the target protein. For example, analysis of the individual ligand clusters (described in more detail below in connection with FIGS. 4, 5A, and 5B) can be used to construct ligand templates. The ligand template can include atoms, functional groups, characteristics, or the like, that are predicted to cause a ligand to have a strong binding affinity to the target protein. In another example, analysis of the ligand clusters can be used to predict putative binding sites on the target protein. In yet another example, information such as the interactions between ligands and proteins in each template, along with the structural alignment of the templates to the target protein, can be used to determine the amino acid residues belonging to the target protein that are most likely to be involved in binding. As discussed previously, information about the molecular function of the target protein (e.g., binding sites, residues involved in ligand binding, a template including characteristics indicative of a ligand that will bind to the protein, or the like) can be used to identify and rank compounds within a large library (e.g., as library of one million drug-like small molecules) based on the predicted ability of the compounds to bind to the target protein. This process can include a comparison of the similarity of the library compounds and the target ligand. This automated computational screening can be fast and can provide a much smaller subset (e.g., 500, 1000, or the like) of compounds that are expected to bind to the target protein.

Since some compounds exhibit low binding specificity, when screening a large compound library, it can be beneficial to not only identify compounds that have high affinity for the target protein, but also have low affinity for other proteins associated with the species of origin of the target protein. In this way, the predicted beneficial affects of binding to the target protein are maximized, while minimizing the side effects that may be caused by binding to other proteins. To accomplish this, the entirety of known proteins of a species can be analyzed using the template-based system and one or more ligand templates can be constructed for each protein. The compounds expected to bind to the target protein can then be compared to all the ligand templates to determine the binding specificity of the compounds. Compounds can be advantageously chosen so as to maximize effectiveness (e.g., have high binding affinity to the target protein) and minimize side effects (e.g., have low binding affinity to the other proteins associated with the species.

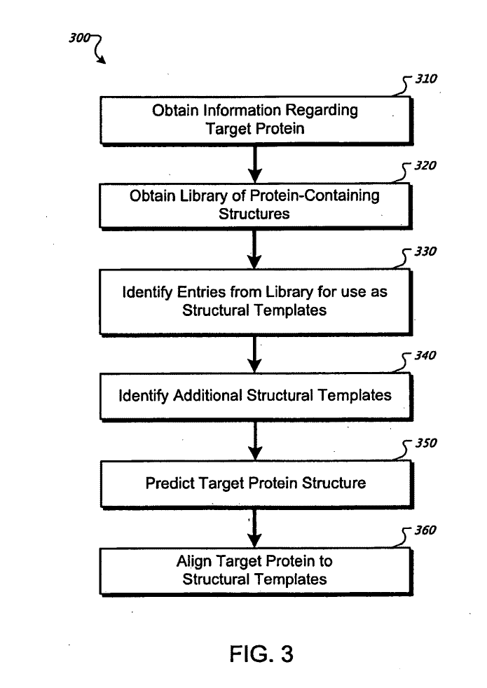

Referring now to FIG. 3, a process 300 can be performed by a template-based system, such as including a suitably configured computer, to determine one or more structural templates for use with a target protein and to subsequently align the structural templates to a predicted structure of the target protein. In some embodiments, the process 300 is exemplary of operations that can be performed by the template-based system during operations 110 -130 (described in connection with FIG. 1). Referring to the process 300, in operation 310, the template-based system can obtain a target amino acid sequence (e.g., the amino acid sequence of a target protein, a portion of a target protein, or the like) to which ligand binding information is desired. The process 300 can execute operation 320, causing the system to obtain a library of information regarding proteins. For example, the system can obtain the entire non-redundant protein library from the PDB, including proteins, bound ligands, structural information, and the like. The protein library can be previously downloaded and stored in the system (e.g., on a hard drive) for access by the system.

In some embodiments, the process 300 can execute operation 330, causing the system to analyze the protein library to identify proteins within the library that can be used as structural templates. For example, the PROSPECTOR_{—}3 (Skolnick, J., D. Kihara, and Y. Zhang, Development and large scale benchmark testing of the PROSPECTOR_{—}3 threading algorithm, Proteins, (2004), 56(3): p. 502-18.) threading algorithm can be used to identify proteins within the library that have a z-score value of greater-than or equal-to 4. In some embodiments, protein files containing no bound ligands, protein files containing multiple bound ligands, or the like, are excluded from use as templates. During operation 340, the template-based system can identify additional templates by searching the library (or other libraries) for homologues (either evolutionary close or distant) of the existing templates.

In some embodiments, during operation 350, the template-based system can predict a structure for the target protein using, for example, the structural templates identified during operations 330-340. For example, the structure prediction can be performed using comparative modeling algorithms, threading alignments, and the like, such as those used by the structure prediction tools MODELLER (Sali, A. and T. L. Blundell, Comparative protein modelling by satisfaction of spatial restraints. J Mol Biol, 1993. 234(3): p. 779-815.), TASSER (Zhang, Y., A. K. Arakaki, and J. Skolnick, TASSER: an automated method for the prediction of protein tertiary structures in CASP6. Proteins, 2005. 61 Suppl 7: p. 91-8.), SWISSPROT (http://www.expasy.ch/sprot/), or the like. The result of these algorithms is a predicted three-dimensional structure of the target protein. During operation 360, the template-based system can align the individual structural templates to the predicted structure of the target protein using structural alignment algorithms such as those used by TM-Align (Zhang, Y and J. Skolnick, TM-align: a protein structure alignment algorithm based on the TM-score. Nucleic Acids Res, 2005. 33(7): p. 2302-9.). Algorithms such as these optimally superimpose two or more proteins based on, for example, the RMSD of the proteins' backbone atoms. The resulting superimposed proteins can be used to determine similarity, superimpose ligands found in the templates onto the target protein, or the like.

Referring now to FIG. 4, a process 400 can be performed by a template-based system, such as including a suitably configured computer, to predict aspects of the molecular function of a target protein, such as putative binding sites of the target protein and characteristics of a ligand predicted to bind with a high affinity to the target protein. In some embodiments, the template-based system can execute the process 400 to identify and rank putative binding sites within a target protein. The putative binding sites can be active sites within the target protein that are predicted to bind to ligands, such as small molecules, metal ions, co-factors, or the like. In some embodiments, at least a portion of the process 400 is exemplary of operations that can be performed by the template-based system during operation 140 (described in connection with FIG. 1). Referring to the process 400, in operation 410, the template-based system can execute operation 410, causing the template-based system to obtain a target amino acid sequence and structure to which ligand binding information is desired. In some embodiments, the process 400 can execute operation 420, causing the system to obtain one or more structural templates that have been aligned to the target protein obtained in operation 410. Exemplary structural templates can be identified and aligned by the process 300 described in connection with FIG. 3. The individual structural templates can contain not only chemical and structural information regarding a protein, but also chemical and structural information about bound ligands. The process 400 can execute operation 430, causing the system to organize the ligands belonging to the structural templates into one or more clusters that represent putative binding sites. For example, when superimposing the templates onto the target protein, the ligands included in the templates are also superimposed onto the target protein. When superimposed, some ligands may be located within close proximity to each other, while other ligands may be found in different locations around the protein. To identify putative binding sites, the system can “cluster” the ligands using a cut-off RMSD (root mean squared deviation) value (e.g., 8 Å). In this example, ligands that fall within 8 Å of each other are considered part of a single cluster. An example of this clustering can be seen in FIGS. 5A-5B. FIG. 5A depicts a target protein 500 that has had multiple structural templates, including bound ligands, superimposed over it. For clarity, the proteins included in the structural templates are not shown, leaving only the structure of the target protein and the ligands 510 contained in the structural templates.

Referring now to FIG. 5B, showing only the bound ligands from the templates reveals the clustering of the ligands. Individual clusters can contain one or more ligands in close proximity. For example, a cluster 520 can include a singleton ligand 521. A cluster 530 can include ligands 531 and 532 (which almost completely overlap) and ligands 541, 542, and 543 can be assigned to a cluster 540. Each one of the individual ligand clusters 520, 530, and 540 can be indicative of an individual binding site on the target protein 500 (FIG. 5A). While the example depicted in FIGS. 5A-5B show one, two, and three ligands per cluster, it should be understood that ligand clusters can contain more than three ligands (e.g., more than 10 ligands, more than 25 ligands, more than 125 ligands, 150 ligands, or the like).

Referring again to FIG. 4, the process 400 can execute operation 440, causing the system to rank the putative binding sites by confidence level. The putative binding sites can be ranked according to the number of structural templates determined in operation 430 to share a common binding pocket (referred to as cluster multiplicity), where a greater number of structural templates associated with a binding site implies a higher ranking. In some embodiments, the number of structural templates associated with a putative binding site can be used to classify each individual putative binding site of the target protein into one of three confidence index groups. Binding sites that are associated with more than 125 structural templates can be classified as having a high level of confidence, binding sites that are associated with between 25 and 125 (inclusive) structural templates can be classified as having a medium level of confidence, and binding sites that are associated with less than 25 templates can be classified as having a low level of confidence. These three confidence level groups can correlate well with the accuracy of the predicted binding site.

In some embodiments, during operation 450, ligands contained in clusters (such as the cluster 530 depicted in FIG. 5B) can be analyzed to determine atoms that are equivalent across the ligands belonging to an individual cluster. For example, the ligands can be analyzed using a chemical compound-matching algorithm, such as SIMCOMP (Hattori, M., et al., “Heuristics for chemical compound matching”, Genome Inform., 2003. 14: p. 144-53), that provides atom equivalences. For example, a similarity cutoff (SC) of 0.7 can be used in SIMCOMP to identify similar compounds containing equivalent atoms. These equivalent atoms can be projected into functional groups, during operation 460, into, for example, one of the set of 17 functional groups listed in table 1. It would be understood by one skilled in the art that the functional groups in Table 1 are not unique for this purpose and that other smaller or larger sets of functional groups, containing some or all of the groups in Table 1 in conjunction with one or more other groups, would be suitable. In operation 470, functional groups that are conserved across a high percentage of ligands in a cluster can be used to construct a ligand template, indicative of a ligand predicted to have a high binding affinity for the target protein. For example, the functional groups that are equivalent in at least 90% of the ligands in a single cluster can identify an anchor substructure. In this example, the anchor substructure can be defined as a maximum set of conserved ligand functional groups. Each anchor substructure can define a ligand template that is indicative of compounds that are predicted to bind to the target protein. This process can be repeated for any or all of the putative binding sites determined during operation 430. In some embodiments, ligand templates can be constructed for a subset of the putative binding sites based on the results of the categorization and ranking performed in operation 440. For example, ligand templates can be constructed for each of the putative binding sites predicted with a high or medium level confidence level as determined in operation 440.

TABLE 1
Exemplary functional groups used for
the projection of ligand heavy atoms.

| Number | Description | Symbol/formula |
|---|---|---|
| 1 | Aromatic rings | mono-, heterocyclic |
| | | five-, six-membered |
| 2 | Ether | —C—O—C— |
| 3 | Thioether | —C—S—C— |
| 4 | Carbonyl | >C═O |
| 5 | Thiocarbonyl | >C═S |
| 6 | Halogene | —Cl; —Br; —F; —I |
| 7 | Guanidine | —NHC(NH₂)NH |
| 8 | Amide | —CONH— |
| 9 | Carboxyl | —COOH |
| 10 | Amine (primary, secondary, tertiary) | —NH₂; >NH; >N— |
| 11 | Phosphate | —PO₄ |
| 12 | Sulphate | —SO₄ |
| 13 | Nitro group | —NO₂ |
| 14 | Metals | Fe; Zn; Mg; Ca |
| 15 | Hydroxyl group | —OH |
| 16 | Thiol group | —SH |
| 17 | A fragment of an aliphatic chain | —(C—C)ₓ—; |
| | composed of at least 2 carbons | —(C═C)ₓ—; |
| | not connected to groups 7-16 | —(C≡C)ₓ— |

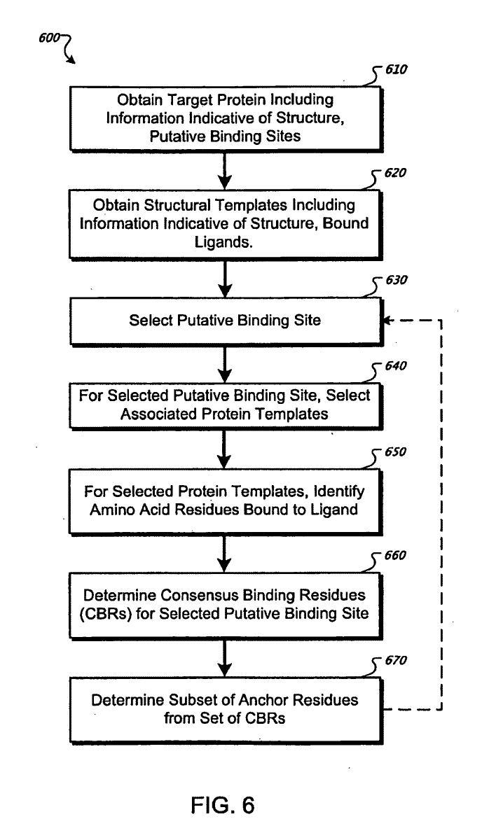

Referring now to FIG. 6, a process 600 can be performed by a template-based system, such as including a suitably configured computer, to predict aspects of the molecular function of a target protein. In some embodiments, the template-based system can execute the process 600 to construct one or more ligand templates that are representative of molecules that are likely to bind to the functional sites of the target protein. In some embodiments, the individual ligand templates can be compared to libraries of compounds to determine which of the compounds is most likely to bind to the functional sites of the target protein. In some embodiments, molecular function information (e.g., consensus binding residues, anchor residues, viz. residues in contact with the ligand anchor region, or the like) can be used to virtually dock ligands to the target protein to predict compounds that have a high affinity for the target protein. Some embodiments can use a combination of these two methods. Referring to the process 600, in operation 610, the template-based system can obtain a target amino acid sequence including information indicative of the structure and one or more putative binding sites of the protein. The structure information can be obtained from a predicted structural model, an experimental target structure, or the like.

In some embodiments, the process 600 can execute operation 620, causing the system to obtain one or more structural templates that have been aligned to the structure of the target protein obtained in operation 610. Exemplary structural templates can be identified and aligned by the process 300 described in connection with FIG. 3. The individual structural templates can contain not only chemical and structural information regarding a protein, but also chemical and structural information about bound ligands. The process 600 can execute operation 630, causing the system to select an individual putative binding site within the target protein from the one or more putative binding sites obtained during operation 610.

In some embodiments, the process 600 can execute operation 640, causing the system to select the protein templates that are associated with the putative binding site selected during operation 630. For example, one or more binding sites within the target protein can be predicted and ranked by the process 400 described in more detail in connection with FIG. 4 and the top ranked binding site can be chosen during operation 630. In some embodiments, the binding sites can be predicted based on the results of aligning ligand-containing structural templates to the target protein and subsequently clustering the ligands (described in more detail in connection with the operations 420 and 430 of the process 400). The structural templates that include ligands that cluster into the currently chosen putative binding site are selected during operation 640.

In some embodiments, the process 600 can execute operation 650, causing the system to identify the amino acid residues, in each of the individual structural templates selected during operation 640, which are contacting the ligand contained in the same structural template. Individual amino acid residues are considered to be contacting a ligand if any heavy atom (e.g., non-hydrogen atom) of the residue is in contact with a ligand heavy atom. Interatomic contact can be established using surface-based algorithms, such as LPC (Sobolev, V., et al., Automated analysis of interatomic contacts in proteins. Bioinformatics, 1999. 15(4): p. 327-32.). During operation 660, the system determines consensus binding residues (CBRs), which are defined as residues contacting a ligand in at least 25% of the structural templates selected during operation 640.

In some embodiments, during operation 670, the system can determine anchor residues of the target protein. The anchor residues can be a subset of CBRs that are in contact with the anchor region of the ligand and have low sequence variability. For example, Shannon's information entropy (Eq. 1) can be used to determine the sequence variability at a particular position in a target protein.

$$Eq . {s}_{l} = - {\sum }_{k = 1}^{7} {p}_{k} {log}_{2} ( {p}_{k} ) \qquad Eq . 1$$

where pₖ is the probability that the i-th residue position is occupied by an amino acid of class k. Here, the class k of an amino acid can be determined from table 2. Residues with an sᵢ value greater than 0.5 are further classified as anchor CBRs, while residues with an sᵢ value equal to or less than 0.5 (all other CBRs) are further classified as non-anchor CBRs. The process 660 can optionally return to operation 630 where another putative binding site is chosen for further analysis by the system during operations 640-670.

TABLE 2
Amino acid classification used in sequence entropy analysis.

| Class | Residues |
|---|---|
| 1 | Ala, Val, Leu, Ile, Met, Cys |
| 2 | Gly, Ser, Thr |
| 3 | Asp, Glu |
| 4 | Asn, Gln |
| 5 | Arg, Lys |
| 6 | Pro, Phe, Tyr, Trp |
| 7 | His |

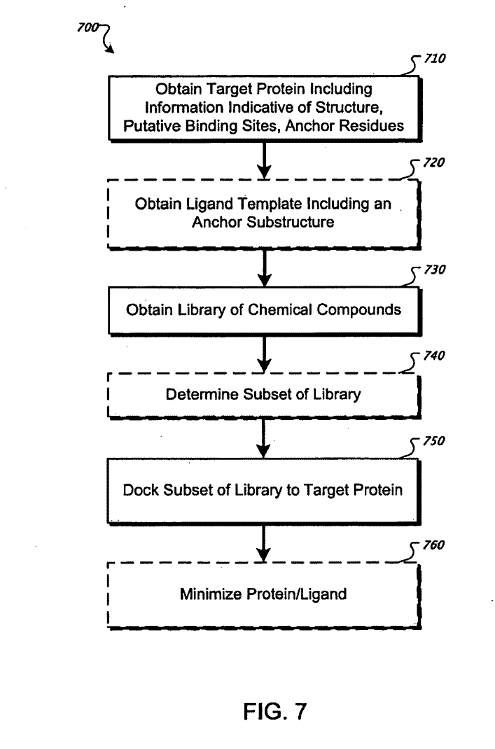

Referring now to FIG. 7, a process 700 can be performed by a template-based system, such as including a suitably configured computer, identify compounds that are predicted to bind to a target protein. For example, the system can screen a database of compounds and determine a subset of these compounds that are predicted to bind to the target protein. Furthermore, the compounds contained in the subset of compounds can be ranked based on their predicted affinity for the target protein. In another example, ligands from a library can be virtually docked to a subset of the amino acid residues belonging to the target protein, such as the anchor residues described previously in connection with FIG. 6. Referring to the process 700, in operation 710, the template-based system can obtain an amino acid sequence for a target protein including information indicative of the structure of the protein, one or more putative binding sites of the protein, anchor residues of the protein, and the like. For example, the template-based system can obtain information indicative of the protein structure from operation 350 of the process 300, described in greater detail in connection with FIG. 3. The template based system can obtain information about putative binding sites from the process 400, described in greater detail in connection with FIG. 4, and information about anchor residues from the process 600, described in greater detail in connection with FIG. 6.

In some embodiments, the process 700 can execute operation 720, causing the system to obtain a ligand template including an anchor substructure such as can be obtained from the process 400, described previously in connection with FIG. 4. During operation 730, the system can obtain a library of chemical compounds, such as those available from the KEGG compound library (Kanehisa, M. and S. Goto, KEGG: kyoto encyclopedia of genes and genomes, Nucleic Acids Res., 2000. 28(1): p. 27-30.), the MDL Data Drug Report (http://www.mdl.com/), the Asinex Platinum Collection of lead-like compounds (http://www.asinex.com/), or the like. The process 700 can optionally execute operation 740 causing the system to determine a subset of the chemical library that includes chemical compounds predicted to bind with a high affinity to the target protein. The chemical compounds in the determined subset can be, for example, screened later screened using other drug discovery techniques, used as lead compounds in a drug development process, administered as part of a therapeutic treatment for a disease, and the like. For example, compounds predicted to bind with a high affinity to HIV-1 protease can be further tested using other drug discovery techniques, modified as part of a drug development process, tested on humans, and the like.

In some embodiments, the process 700 can execute operation 750, causing the template-based system to virtually dock some or all of the chemical compounds contained in the library to the target protein. For example, the template-based system can dock the entire contents of a chemical library to the target protein to predict which compounds contained within the library bind to the protein with a high affinity. In other examples, the system can dock the subset of proteins determined during operation 740 to the target protein.

During operation 760, the system can optionally perform energy minimizations on protein-ligand complexes containing ligands predicted to bind with a high affinity to the protein. For example, the protein-ligand complexes determined during operation 750 can be refined by energy minimization in AMBER 8 (Pearlman, D. A., et al., “AMBER, a computer program for applying molecular mechanics, normal mode analysis, molecular dynamics and free energy calculations to elucidate the structures and energies of molecules” Comp. Phys. Commun., 1995. 91: p. 1-41.).

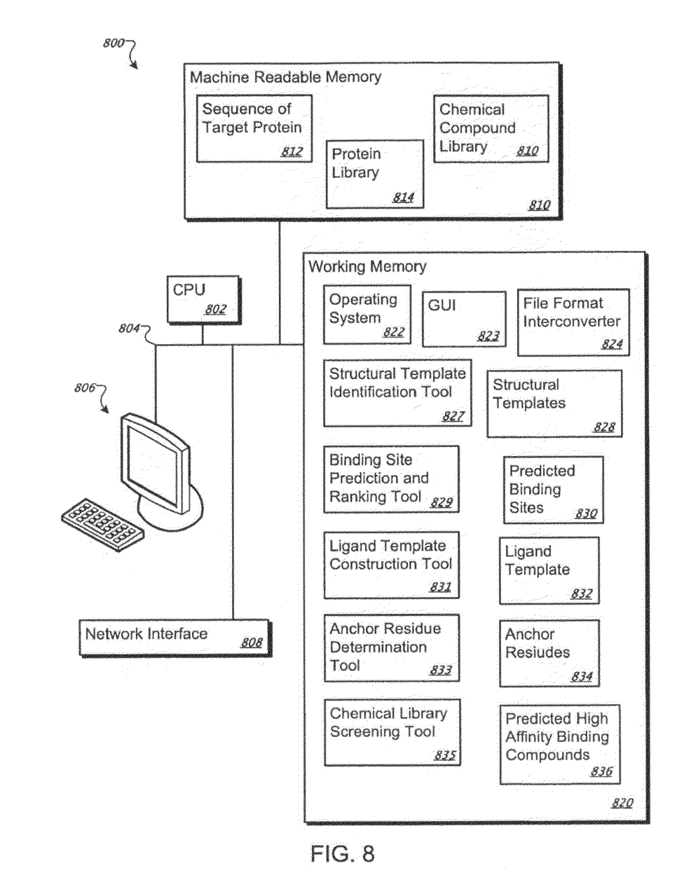

Referring now to FIG. 8, a computer system 800 on which macromolecular structure template-based system as described herein may be carried out can include one or more central processing units 802 for processing machine readable data coupled via a bus 804, to a user interface 806, a network interface 808, a machine readable memory 810, and a working memory 820. The machine readable memory 810 can include a data storage material encoded with machine readable data, wherein the data comprises, for example, one or more target protein sequences 812, one or more protein libraries 814, and one or more chemical compound libraries 816.

Working memory 820 can store an operating system 822, a GUI 823, and file format converters 824. The working memory 820 can store data associated with the process of the template-based system, such as one or more structural templates 828, one or more predicted binding sites 830, one or more ligand templates 832, one or more anchor residues 834, and one or more compounds 836 predicted to have a high affinity for the target protein 812. The computer system 800 can also include instructions for processing machine readable data including one or more structural structural template identification tools 827, one or more binding site prediction and ranking tools 829, one or more ligand template construction tools 831, one or more anchor residue determination tools 833, and one or more chemical library screening tools 835.

The computer system 800 may be any of the varieties of laptop or desktop personal computer, or workstation, or a networked or mainframe computer or super-computer, which would be available to one of ordinary skill in the art. For example, computer system 800 may be an IBM-compatible personal computer, a Silicon Graphics, Hewlett-Packard, Fujitsu, NEC, Sun or DEC workstation, or may be a supercomputer of the type formerly popular in academic computing environments. Computer system 800 may also support multiple processors as, for example, in a Silicon Graphics “Origin” system, or a cluster of connected processors.

The operating system 822 may be any suitable variety that runs on any of computer systems 800. For example, in one embodiment, operating system 822 is selected from the UNIX family of operating systems, for example, Ultrix from DEC, AIX from IBM, or IRIX from Silicon Graphics. It may also be a LINUX operating system. In other embodiments, operating system 822 may be a VAX VMS system. In still other embodiments, the operating system 822 can be a DOS operating system or a Windows operating system, such as Windows 3.1, Windows NT, Windows 95, Windows 98, Windows 2000, Windows XP, or Windows Vista. In yet other embodiments, operating system 822 is a Macintosh operating system such as MacOS 7.5.x, MacOS 8.0, MacOS 8.1, MacOS 8.5, MacOS 8.6, MacOS 9.x and MacOS X.

The graphical user interface (“GUI”) 823 is used, for example, for displaying predicted binding sites on a protein (e.g., the predicted binding sites 830), and/or listing compounds that are predicted to have a high affinity for the target protein, on user interface 806. The user-interface 806 may comprise input and output devices such as a keyboard, mouse, touch-screen, display screen, trackpad, scanner, printer, or projector.

The network interface 808 may optionally be used to access, for example, one or more protein libraries and/or chemical compound libraries stored in the memory of one or more other computers. One or more aspects of the template-based methods described herein may be carried out with commercially available programs which run on, or with computer programs that are developed specially for the purpose and implemented on, computer system 800. Exemplary commercially available programs can include spreadsheet software (e.g., Excel), and proteomics tools (e.g., TM-Align (Zhang, Y. and J. Skolnick, TM-align: a protein structure alignment algorithm based on the TM-score. Nucleic Acids Res, 2005. 33(7): p. 2302-9.), PROSPECTOR_{—}3 (Skolnick, J., D. Kihara, and Y. Zhang, Development and large scale benchmark testing of the PROSPECTOR_{—}3 threading algorithm. Proteins, 2004. 56(3): p. 502-18.), MODELLER (Sali, A. and T. L. Blundell, Comparative protein modelling by satisfaction of spatial restraints. J Mol Biol, 1993. 234(3): p. 779-815.), TASSER (Zhang, Y., A. K. Arakaki, and J. Skolnick, TASSER: an automated method for the prediction of protein tertiary structures in CASP6. Proteins, 2005. 61 Suppl 7: p. 91-8.), or the like). Alternatively, the template-based methods may be performed with one or more stand-alone programs each of which carries out one or more operations of the template-based system.

### EXAMPLES

### Example 1

### Ligand Anchor Region Features and Use in Ligand Docking

The protocol followed in this example was a direct extension of FINDSITE (Brylinski, M. and J. Skolnick, A threading-based method (FINDSITE) for ligand-binding site prediction and functional annotation. Proc. Natl. Acad. Sci. USA, 2008. 105(1): p. 129-34), a threading-based method for ligand-binding site prediction and functional annotation that detects the conservation of functional sites and their properties in evolutionarily related proteins. FINDSITE is further described in: (Brylinski, M. and J. Skolnick, A threading-based method (FINDSITE) for ligand-binding site prediction and functional annotation. Proc Natl Acad Sci USA, 2008. 105(1): p. 129-34), incorporated herein by reference. FINDSITE identifies ligand-bound template structures from a set of homologous proteins recognized for a given target sequence by the PROSPECTOR_{—}3 (Skolnick, J., D. Kihara, and Y. Zhang, Development and large scale benchmark testing of the PROSPECTOR_{—}3 threading algorithm. Proteins, 2004. 56(3): p. 502-18.) threading approach. In this example, FINDSITE was used with a set of distantly homologous proteins (e.g., <35% target-template sequence identity), and superimposed them onto a target structure with the TM-Align (Zhang, Y. and J. Skolnick, TM-align: a protein structure alignment algorithm based on the TM-score. Nucleic Acids Res, 2005. 33(7): p. 2302-9.) structure alignment algorithm. One or more binding pockets were identified by the spatial clustering of the center of mass of template-bound ligands. These binding pockets were subsequently ranked by the number of ligands that bound to the individual sites.

In this example, the protein-ligand complexed structures used were taken from the Astex diverse set of protein-ligand complexes (Hartshorn, M. J., et al., Diverse, High-Quality Test Set for the Validation of Protein-Ligand Docking Performance. J. Med. Chem., 2007. 50(4): p. 726-741.) used to validate the GOLD docking algorithm (Jones, G, et al., Development and validation of a genetic algorithm for flexible docking. J Mol Biol, 1997. 267(3): p. 727-48.) and from a non-redundant Q-Dock dataset (Brylinski, M. and J. Skolnick, Q-Dock: Low-resolution flexible ligand docking with pocket-specific threading restraints. J Comput Chem, 2008. 29(10): p. 1574-1588.), both of which are high-quality ligand-bound protein structures determined by X-ray crystallography. In the Astex dataset, complexes in which the binding site is formed by more than one protein chain were excluded. In the Q-Dock set, proteins with >35% sequence identity to any protein in the Astex set were excluded. Additionally, proteins that did not meet the following criteria were excluded: proteins for which at least 5 ligand-bound weakly homologous threading templates were identified by protein threading and the binding pocket was predicted by FINDSITE within 4.5 A from the bound ligand. About 67% of protein targets met these criteria and were not excluded.

In addition to the crystal structures used as structures of the target proteins, the performance of FINDSITE^{LHM} was evaluated. FINDSITE^{LHM} is an algorithm that employs FINDSITE (Brylinski, M. and J. Skolnick, A threading-based method (FINDSITE) for ligand-binding site prediction and functional annotation. Proc Natl Acad Sci USA, 2008. 105(1): p. 129-34) to predict the ligand binding site in the protein followed by docking of the anchor substructure of the ligand of interest to the predicted pose of the ligand anchor region extracted from FINDSITE identified templates. FINDSITE^{LHM} was evaluated in ligand docking using weakly homologous protein models (proteins whose closest template sequence identity to the target protein was <35%). In this example, the restriction of using proteins whose closest template sequence identity to the target protein was <35% was employed for benchmarking purposed only. Non-benchmarking applications of FINDSITE^{LHM} would not require this restriction. Here, the Dolores dataset of 205 protein models (Wojciechowski, M. and J. Skolnick, Docking of small ligands to low-resolution and theoretically predicted receptor structures. J Comput Chem, 2002. 23(1): p. 189-97.), generated by the protein structure prediction protocol TASSER (Zhang, Y., A. K. Arakaki, and J. Skolnick, TASSER: an automated method for the prediction of protein tertiary structures in CASP6. Proteins, 2005. 61 Suppl 7: p. 91-8.), was considered.

To predict the binding pocket, the PROSPECTOR_{—}3 (Skolnick, J., D. Kihara, and Y. Zhang, Development and large scale benchmark testing of the PROSPECTOR_{—}3 threading algorithm. Proteins, 2004. 56(3): p. 502-18.) threading algorithm was used, for a given amino acid sequence, to identify weakly homologous structure templates. In this example, templates with >35% sequence identity to target protein were excluded, leaving weakly homologous structure templates. Structures that bind a ligand were identified by FINDSITE (Brylinski, M. and J. Skolnick, A threading-based method (FINDSITE) for ligand-binding site prediction and functional annotation, Proc. Natl. Acad. Sci. USA, 2008. 105(1): p. 129-34) and superimposed onto the reference crystal structure by TM-Align (Zhang, Y. and J. Skolnick, TM-align: a protein structure alignment algorithm based on the TM-score. Nucleic Acids Res, 2005. 33(7): p. 2302-9.). FINDSITE employed an average linkage clustering procedure to cluster the centers of mass of template-bound ligands to detect putative binding sites and then ranks them by the number of ligands.

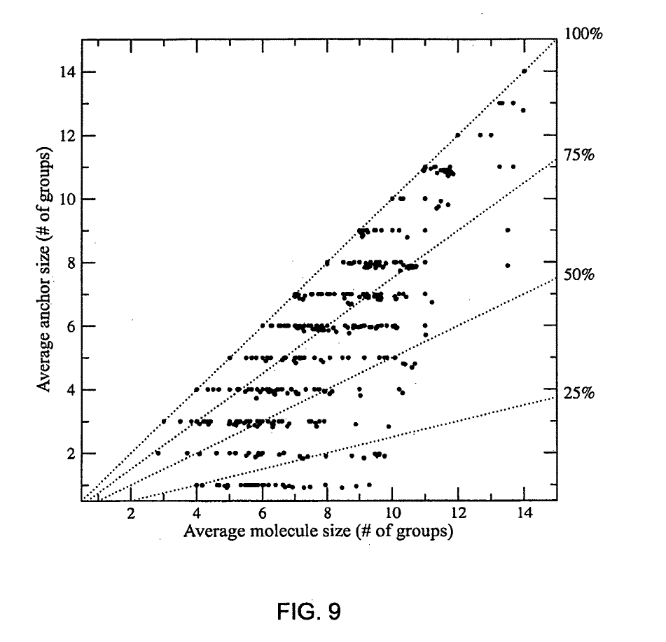

To perform ligand clustering, template-bound ligands that occupy top-ranked predicted binding pockets were clustered using a SIMCOMP (Hattori, M., et al., Heuristics for chemical compound matching. Genome Inform, 2003. 14: p. 144-53.) similarity (SC) cutoff value of 0.7. SIMCOMP (Hattori, M., et al., Heuristics for chemical compound matching. Genome Inform, 2003. 14: p. 144-53.) is a chemical compound-matching algorithm that provides atom equivalences. Each cluster of ligand molecules was used to detect an anchor substructure. First, the anchor substructure size was examined relative to the average molecule size. Referring to FIG. 9, applying the approach to the set of 711 ligand-protein complexes defined previously, in most cases at least 50% of a ligand is comprised of an anchor region whose functional groups are conserved in >90% of the template ligands. Those ligands belonging to clusters in which the anchor region was smaller than 50% were mostly comprised of short oligosaccharides with a sugar monomer identified as a common substructure. The inclusion of these ligands also explains the high standard deviation in the average molecule size. For some difficult cases, the graph isomorphism analysis applied here did not provide sufficient atomic equivalences to recognize a common substructure. Oppositely, targets near the diagonal have an anchor equivalent to the average molecule size and represent strongly conserved ligands with little chemical variability. Included in this list of targets are hemes, as well as targets with poor statistics (e.g., few identified templates complexed with very similar ligands). For the majority of the targets, a well-defined anchor substructure with a co-occurring variable region was detected.

To define the anchor substructure, the equivalent atom pairs provided by SIMCOMP (Hattori, M., et al., Heuristics for chemical compound matching. Genome Inform, 2003. 14: p. 144-53.) were projected onto ligand functional groups. Here, the atoms were projected onto the set of 17 functional groups listed in table 1. The anchor substructure was defined as a maximum set of conserved functional groups present in at least 90% of the ligands from a single cluster.

Due to the chemical conservation of the anchor substructure as well as the strong structural conservation of the binding mode across evolutionarily distant proteins, it can be predicted that the residues in contact with the ligand anchor substructures are more conserved than average. The term consensus binding residues (CBRs) was defined as residues contacting a ligand in at least 25% of the threading templates. The degree of sequence and structure conservation was then calculated for the CBRs. This criterion was previously found to maximize the overlap between predicted and observed binding residues and provided sufficient statistics to calculate sequence and structural features of binding residues. A probability threshold was used to define anchor/non-anchor CBRs based on the protein-ligand contacts extracted from the threading templates. The probability of a residue to be an anchor residue corresponded to the fraction of contacts formed by all residues in the equivalent position in the template structures with anchor functional groups of bound ligands. Differences in the degree of sequence and structure conservation between anchor and non-anchor CBRs were calculated by increasing the probability threshold from 0.1 to 0.9 using Student's t-test for independent samples.

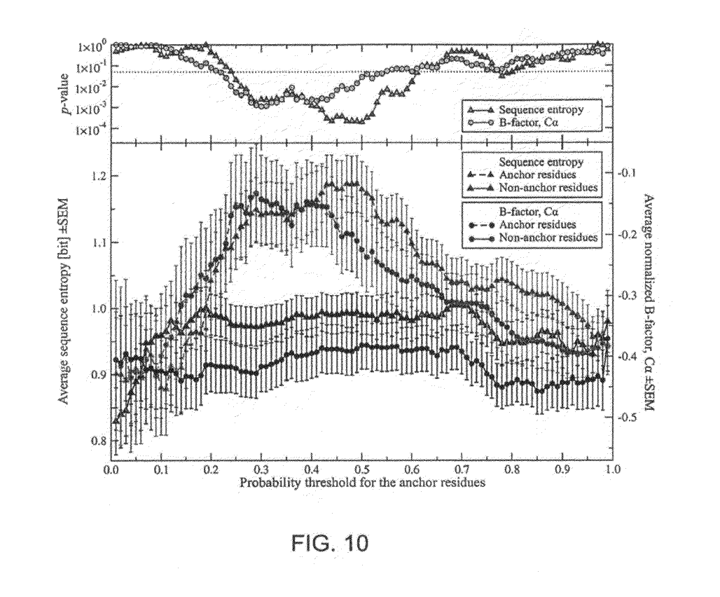

In this example, Shannon's information entropy (Eq. 1) was used to measure the sequence variability, sᵢ, at a particular position, i, in a target protein wherein pₖ is the probability that the i-th residue position is occupied by an amino acid of class k, as listed in table 2. Amino acids were grouped by their chemical similarity, since the chemical conservation of anchor functional groups requires residues of a particular class to be in contact more often than other residues. Analysis of the sequence entropy revealed a significantly higher sequence conservation of residues in contact with the anchor functional groups than those in contact with ligand variable regions (see FIG. 10). The maximum difference between the two classes was observed for an anchor CBR's probability threshold of 0.5, (i.e., a CBR is considered to be an “anchor” residue if the residues in the equivalent position in evolutionarily related proteins are more often found in contact with anchor functional groups than with variable groups of the bound ligands).

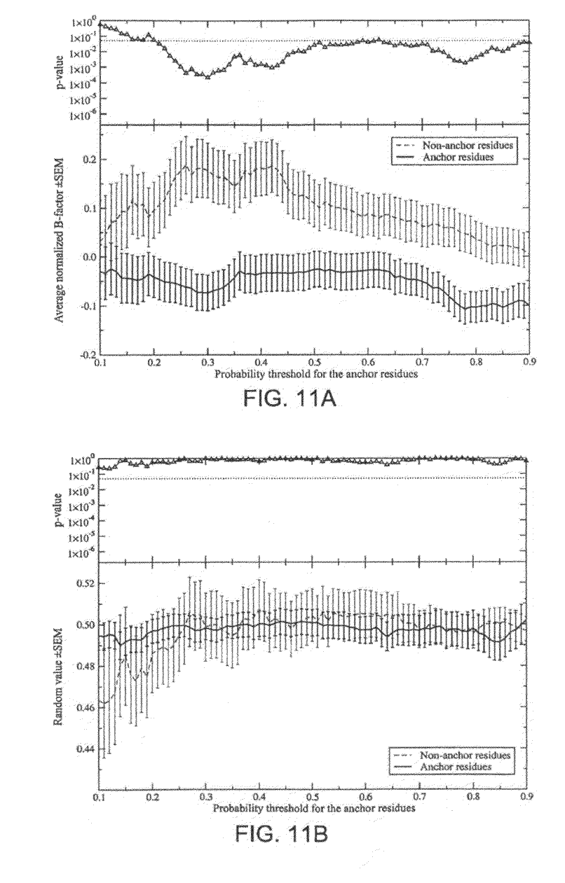

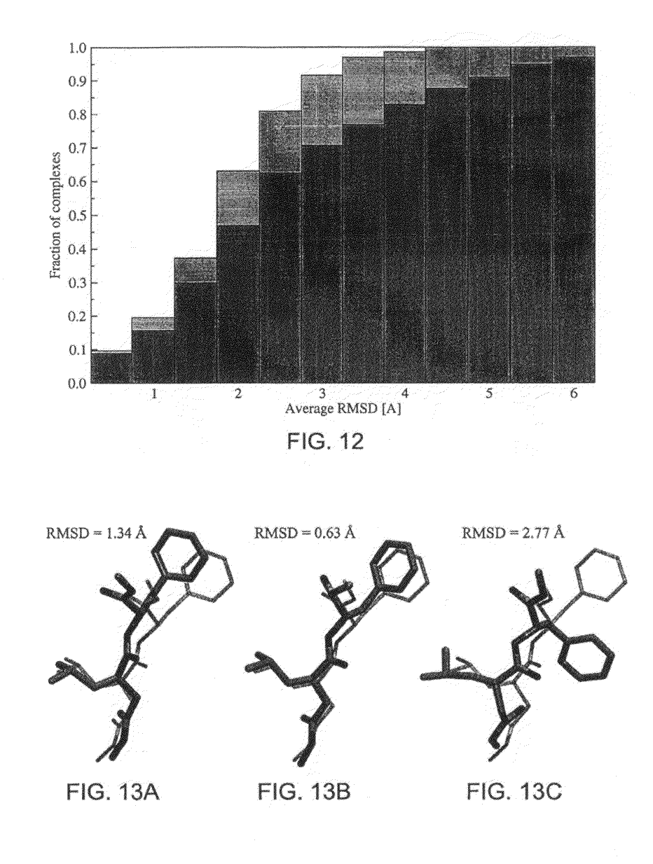

Having identified the anchor substructure, the structural conservation of the anchor substructures binding mode was investigated. The structure features of CBRs were analyzed in terms of the experimental B-factors, which reflect local mobility. Raw experimental B-factors were extracted from the PDB. Prior to normalization, outliers were detected and removed using the median-based method. To compare B-factors extracted from various protein structures solved in different conditions and at different resolution, a normalization procedure was applied. The results presented in FIG. 10, FIG. 11A, and FIG. 11B show the differences in the average normalized B-factor between anchor and non-anchor CBRs calculated for the Cα atoms (FIG. 10) as well as for the side chain heavy atoms (FIG. 11A). The conservation of the anchor-binding pose can be explained by the relatively lower B-factors observed for the residues in spatial proximity to the anchor functional groups. Here, the maximum difference between the average B-factors for anchor and non-anchor CBRs was found for a probability threshold of 0.3. CBRs that form contacts with anchor substructures of bound ligands in at least 30% of the template structures were significantly less flexible than other binding residues. These results differ significantly from random (FIG. 11B). FIG. 12 shows the histogram of the average pairwise RMSD among the anchor substructures upon global superposition of the template proteins. Note that the properties of the native ligand are not used in any way to identify the anchor region's properties. Clearly, the average pairwise RMSD is in most (˜60%) cases <2.5 Å.

The docking procedure implemented in FINDSITE^{LHM} employed the superposition of the ligand to be docked to the target protein and the target ligand onto the consensus binding pose of the identified anchor substructure of the ligand as oriented in the protein binding pocket. The consensus binding pose was defined as the anchor conformation averaged over the seed compounds (the largest set of compounds that have their anchor substructures within a 4 Å RMSD from each other). In this example, no structural information from the crystal structure of the target complex was used to derive the consensus binding pose of an anchor or to identify the anchor itself. If multiple anchor substructures are predicted for a given target, the one derived from the cluster of template-bound ligands with the highest average chemical similarity to the target ligand was selected, as assessed by its SIMCOMP (Hattori, M., et al., Heuristics for chemical compound matching. Genome Inform, 2003. 14: p. 144-53.) score. This procedure attempts to maximize the coverage of the selected anchor. In addition, if atom equivalences for non-anchor atoms can be established between the target ligand and any of template-bound ligands, their positions are also included in the set of the reference coordinates. By including additional coordinates, substantially correct positions of ligand variable groups can provide a good initial conformation for post-docking refinement. If none of the identified anchor substructures was covered by the target ligand, the ligand was randomly placed in the predicted pocket. Ligand flexibility was addressed by superpositioning multiple conformations of the target ligand. The conformation that was superposed onto the reference coordinates with the lowest RMSD (when compared to the predicted anchor pose) was selected as the final model. In some examples, models of the protein-ligand complexes generated by FINDSITE^{LHM} were optionally refined by energy minimization in AMBER 8 (Pearlman, D. A., et al., Comp. Phys. Commun., 1995. 91: p. 1-41.).

Models of protein-ligand complexes generated by FINDSITE^{LHM} were refined by simple energy minimization in AMBER 8 (Pearlman, D. A., et al., Comp Phys Commun, 1995. 91: p. 1-41.). The Amber force field 03 was used for proteins and the general Amber force field (GAFF) was used for ligands. The system was neutralized using chloride or sodium ions, if necessary. Protein atoms were fixed while the ligand conformation was energy minimized in 1000 cycles of the steepest-descent procedure followed by 1000 cycles of the conjugate gradient procedure.

The performance of FINDSITE^{LHM} was then compared to that of other ligand docking approaches such as AutoDock, LIGIN and Q-Dock. In this example, AutoDock 3 (Morris, G. M., et al., Automated docking using a Lamarckian genetic algorithm and an empirical binding free energy function, Journal of Computational Chemistry, 1998. 19(14): p. 1639-1662.) was used in the flexible ligand docking simulations. Input files for both receptors and ligands were prepared using MGL Tools 1.5.2 (Sanner, M. F., Python: a programming language for software integration and development. J Mol Graph Model, 1999. 17(1): p. 57-61.). A grid spacing of 0.375 Å was used, with the box dimensions depending on the target ligand size, such that the ligand's geometric center was not allowed to move more than 7 Å away from the predicted binding pocket center. Each docking simulation consisted of 100 runs of a genetic algorithm (GA) using the default GA parameters. The lowest-energy conformation was taken as the final docking result. For virtual docking simulations using Q-Dock (Brylinski, M. and J. Skolnick, Q-Dock: Low-resolution flexible ligand docking with pocket-specific threading restraints. J Comput Chem, 2008. 29(10): p. 1574-1588.), the protocol for low-resolution ligand docking was followed using Replica Exchange Monte Carlo (MC). Ligand flexibility was accounted for by docking the ensemble of, at most 50, non-redundant (1 Å pairwise RMSD cutoff) discrete ligand conformations; the number of conformations depends on the number of rotatable bonds and the hybridization of bonded atoms. A 7 Å radius docking sphere was used (7 Å is the maximal allowed distance between the ligand's geometric center and the center of the predicted binding pocket). The simulations utilized 16 replicas and consisted of 100 attempts at replica exchange and 100 MC steeps between replica swaps. The final model corresponds to the lowest-energy conformation. For virtual docking simulations using LIGIN (Sobolev, V., et al., Molecular docking using surface complementarity. Proteins, 1996. 25(1): p. 120-9.), the idea of ligand docking using conformational ensembles was adopted to mimic ligand flexibility in LIGIN. For a given target, the same ensemble of multiple ligand conformations as in Q-Dock simulations and FINDSITE^{LHM} was used. The ensembles were docked into the predicted binding site using LIGIN and the docking procedure was repeated 1000 times for each ligand conformer. The final binding mode corresponds to that of the maximal complementarity found in the complete set of ligand conformers. Atom types were assigned using LPC (Sobolev, V., et al., Automated analysis of interatomic contacts in proteins. Bioinformatics, 1999. 15(4): p. 327-32.); no receptor residues were permitted to have steric overlap with the ligand.

Table 3 shows the results obtained for a representative set of 711 ligand-protein complexes (whose target proteins are non homologous) using the crystal structures as the target receptors for ligand docking. To equitably assess the results, the target proteins were divided into three subsets with respect to the coverage of the predicted anchor substructure. The first subset (full coverage) consists of proteins for which a portion of their target ligands covered at least 90% of the functional groups in the predicted anchor substructure. Note that for a given protein, the possibility that multiple anchor substructures could occupy the predicted binding pocket was allowed. Then, the binding mode of the most covered anchor was used as the reference coordinate for the target ligand superposition. As shown in table 3, simple ligand superposition outperformed regular ligand docking approaches if a target ligand matched at least 90% of the anchor substructure.

TABLE 3
Docking results for FINDSITE^{LHM} dataset in terms of ligand
heavy atom RMSD from the crystal structure. The RMSD values
are reported for three subsets comprising ligands with
different ligand anchor region coverage.

| Docking | | | |
|---|---|---|---|
| Algorithm | Full coverage* | Partial coverage^{†} | Low coverage^{‡} |
| Targets^{§} | 522 | 142 | 47 |
| FINDSITE^{LHM¶} | 2.81 ± 2.15 | 4.79 ± 2.33 | 5.08 ± 2.08 |
| FINDSITE^{LHM} + | 2.55 ± 2.28 | 4.70 ± 2.52 | 5.03 ± 2.20 |
| minimization^{∥} | | | |
| AutoDock | 3.12 ± 2.61 | 4.34 ± 2.71 | 3.88 ± 3.15 |
| Q-Dock | 3.26 ± 2.12 | 4.93 ± 2.35 | 4.90 ± 2.21 |
| LIGIN | 4.70 ± 2.59 | 4.86 ± 2.59 | 4.46 ± 2.52 |
| Random | 5.85 ± 1.67 | 5.74 ± 1.58 | 5.02 ± 1.49 |

*Target ligand covers ≧90% of the anchor functional groups.
^{†}Target ligand covers ≧50% and <90% of the anchor substructures.
^{‡}Target ligand covers <50% of the anchor substructures.
^{§}Number of target proteins.
^{¶}Ligand superimposed onto the consensus anchor-binding mode.
^{∥}Superimposed conformation minimized with AMBER 8 (Pearlman, D. A., et al., Comp Phys Commun, 1995. 91: p. 1-41.).

By using all-atom minimization in the Amber force field, the predicted binding mode can be refined to the average RMSD from the crystal structure of ˜2.5 Å. An example of the successful refinement is presented for the human fibroblast collagenase in FIGS. 13A-C. The FINDSITE^{LHM} result is shown in FIG. 13A and the subsequently minimized pose is in FIG. 13B. For comparison, the result from Autodock (Morris, G. M., et al., Automated docking using a Lamarckian genetic algorithm and an empirical binding free energy function. Journal of Computational Chemistry, 1998. 19(14): p. 1639-1662.) is shown in FIG. 13C. The RMSD values of 1.34 Å, 0.63 Å, and 2.77 Å (for the FINDSITE^{LHM} result in FIG. 13A, the AMBER minimized FINDSITE^{LHM} result in FIG. 13B, and the Autodock result in FIG. 13C, respectively) were calculated for heavy atoms with respect to the experimental binding pose.

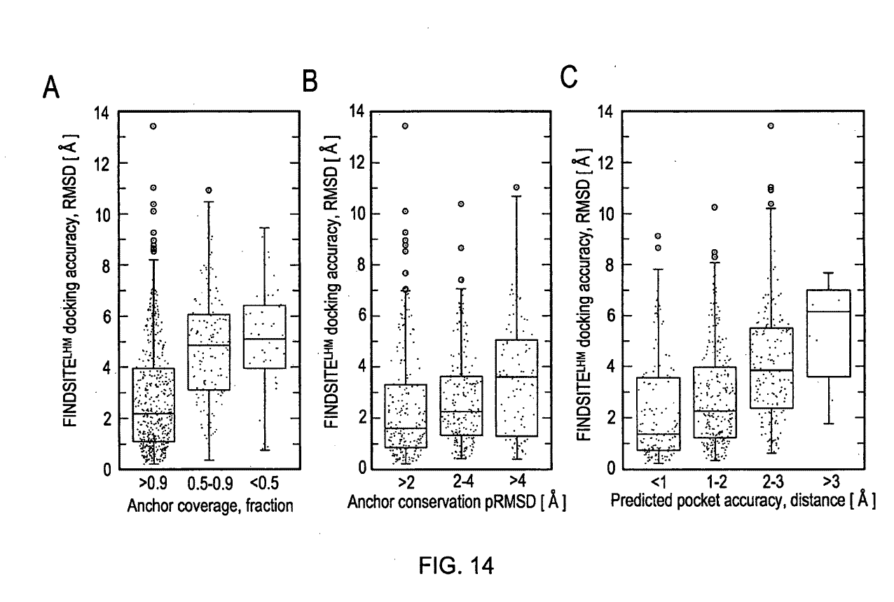

The second subset (partial coverage) comprised target ligands that did not fully cover any of the predicted anchor substructures. Here, the average RMSD of the binding mode predicted by FINDSITE^{LHM} is slightly higher than AutoDock (Morris, G. M., et al., Automated docking using a Lamarckian genetic algorithm and an empirical binding free energy function. Journal of Computational Chemistry, 1998. 19(14): p. 1639-1662.) and is comparable to Q-Dock (Brylinski, M. and J. Skolnick, Q-Dock: Low-resolution flexible ligand docking with pocket-specific threading restraints. J Comput Chem, 2008. 29(10): p. 1574-1588.) and LIGIN (Sobolev, V., et al., Molecular docking using surface complementarity. Proteins, 1996. 25(1): p. 120-9.). However, all of these approaches result in better results than random ligand placement. Finally, if none of the predicted anchor substructures are even partially covered by a target ligand (low coverage), the results of docking using FINDSITE^{LHM} were indistinguishable from random. In the absence of the reference coordinates, as expected, ligands were randomly placed into their binding site. Here, traditional ligand docking approaches, particularly AutoDock (Morris, G. M., et al., Automated docking using a Lamarckian genetic algorithm and an empirical binding free energy function. Journal of Computational Chemistry, 1998. 19(14): p. 1639-1662.), give better results. In addition to anchor structure coverage (see FIG. 14A), the performance of FINDSITE^{LHM} depends on the overall accuracy of binding pocket prediction and the conservation of the anchor-binding mode; this is described further herein in connection with FIGS. 14B-C. Using the fraction of the anchor region that is aligned, for a given ligand, the expected accuracy of binding pose prediction can be predicted without knowing the experimental result.

The average accuracy of the binding mode prediction by FINDSITE^{LHM} decreases with decrease in the degree of the conservation of the anchor substructure (see FIG. 14B). The RMSD of the predicted ligand-binding pose is <2 Å on average for highly conserved anchor substructures whose pairwise RMSD, pRMSD, is <2 Å. For moderately conserved anchor substructures with a pairwise RMSD of 2-4 Å, the RMSD of the predicted ligand-binding mode is <3 Å in most cases. Finally, accompanied by weak (>4 Å) structural conservation of an anchor, docking accuracy drops to >3 Å on average. In addition, the drop off in ligand binding pose prediction correlates with the overall accuracy of binding pocket prediction by FINDSITE (Brylinski, M. and J. Skolnick, A threading-based method (FINDSITE) for ligand-binding site prediction and functional annotation. Proc Natl Acad Sci USA, 2008. 105(1): p. 129-34) (FIG. 14C). The most accurate ligand binding poses were obtained for precisely detected pockets, where the pocket center was predicted within 2 Å from the geometric center of the native ligand. Considering the structural conservation of the derived anchor substructure, its coverage by a target ligand and the FINDSITE (Brylinski, M. and J. Skolnick, A threading-based method (FINDSITE) for ligand-binding site prediction and functional annotation. Proc Natl Acad Sci USA, 2008. 105(1): p. 129-34) confidence index for pocket detection, (all properties which can be calculated without knowledge of the native binding pose), one can roughly estimate the quality of the performance of FINDSITE^{LHM} in ligand binding pose prediction.

Weakly homologous protein models that frequently have significant structural inaccuracies in side-chain and backbone coordinates were considered to be more challenging targets for ligand binding pose prediction. The performance of FINDSITE^{LHM}, AutoDock (Morris, G. M., et al., Automated docking using a Lamarckian genetic algorithm and an empirical binding free energy function. Journal of Computational Chemistry, 1998. 19(14): p. 1639-1662.), Q-Dock (Brylinski, M. and J. Skolnick, Q-Dock: Low-resolution flexible ligand docking with pocket-specific threading restraints. J Comput Chem, 2008. 29(10): p. 1574-1588.) and LIGIN (Sobolev, V., et al., Molecular docking using surface complementarity. Proteins, 1996. 25(1): p. 120-9.) in ligand docking when protein models are used as the target receptors was assessed for the Dolores dataset of 205 protein models (Wojciechowski, M. and J. Skolnick, Docking of small ligands to low-resolution and theoretically predicted receptor structures. J Comput Chem, 2002. 23(1): p. 189-97.). Table 4 presents ligand docking results using crystal structures as well as weakly homologous protein models in terms of the fraction of recovered binding residues and specific native contacts. Considering the complete dataset and receptor crystal structures, the accuracy of FINDSITE^{LHM} is slightly lower than AutoDock and Q-Dock. This is because the predicted anchor substructure was fully covered (≧90%) by the target ligand only for 62.4% of the receptors; partial (≧50% and <90%) and low (<50%) coverage of the anchor substructure was found for 25.4% and 12.2% of the targets, respectively. However, for protein models, FINDSITE^{LHM} recovered more binding residues and specific native contacts than both all-atom docking approaches, AutoDock and LIGIN. As expected, the performance of Q-Dock for protein models was notably higher since it was explicitly designed to deal with structural inaccuracies in the predicted receptor models. We note the high sensitivity of all-atom docking approaches to the quality of the receptor structures; for weakly homologous protein models, essentially, the performance of AutoDock and LIGIN was no better than the random ligand placement into the predicted binding sites. In addition, the all-atom minimization procedure applied to the binding poses predicted by FINDSITE^{LHM} caused a considerable loss of the specific native contacts. An important feature of the FINDSITE/FINDSITE^{LHM} approach for modeling protein-ligand interactions is a reliable confidence index. Considering only the most confident cases for which FINDSITE was likely to predict the binding pocket center with ≦4 Å accuracy (“Easy” targets) and the predicted anchor substructure fully (partially) covered by the target ligand, the fraction of binding residues and specific native contacts recovered by FINDSITE^{LHM} is 0.66 (0.61) and 0.49 (0.43), respectively. These results represent a significant improvement over traditional all-atom docking against modeled receptor structures. In contrast to classical ligand docking approaches, FINDSITE^{LHM} is computationally less expensive; full flexible ligand docking typically requires less than a minute of a CPU time (for docking times, see table 4).

TABLE 4
Docking times for the Dolores dataset. All docking simulations
were performed using a 2.0 GHz AMD Opteron processor. Timings
reported for LIGIN, Q-Dock and FINDSITE^{LHM} include the
pre-docking generation of ligand conformational ensemble
(median: 23 s on a 3.4 GHz P4).

| Docking algorithm | Docking time in seconds* |
|---|---|
| AutoDock^{†} | 11158 (286:1) |
| LIGIN^{‡} | 1623 (42:1) |
| Q-Dock^{§} | 1479 (38:1) |
| FINDSITE^{LHM,¶} | 39 (1:1) |
| FINDSITE^{LHM} + | 48 (1.2:1) |
| minimization^{∥} | |

*Median values are reported; the numbers in parentheses show the ratio of the docking time to FINDSITE^{LHM}.
^{†}100 runs of a genetic algorithm using a grid spacing of 0.375 Å.
^{‡}1000 rounds of optimization for each ligand conformer.
^{§}16 replicas, 100 attempts at replica exchange and 100 MC steps between replica swaps.
^{¶}Superposition of each ligand conformer onto the consensus anchor-binding mode.
^{∥}Including all-atom minimization with AMBER 8 (Pearlman, D. A., et al., Comp Phys Commun, 1995. 91: p. 1-41.).

Previously, a detailed picture of the evolution and diversification of the enzyme function was drawn from the analysis of conservation of substrate substructures in 42 major enzyme superfamilies. Based on graph isomorphism analysis, highly conserved substructures were identified in all substrates of a particular enzyme superfamily. For the remaining substrate substructures, called reacting substructures, substantial variation in chemical properties within the superfamily was found. Here, it was shown that the set of ligands that bind to the common binding site in distantly evolutionarily related proteins contain a set of “anchor” functional groups that are strongly conserved in their chemical features and binding mode, and “variable” regions that account for a specificity toward a particular family member. The highly conserved substructures of the enzyme substrates identified previously frequently overlap with the conserved anchor substructures detected by the threading-based approach in this example.

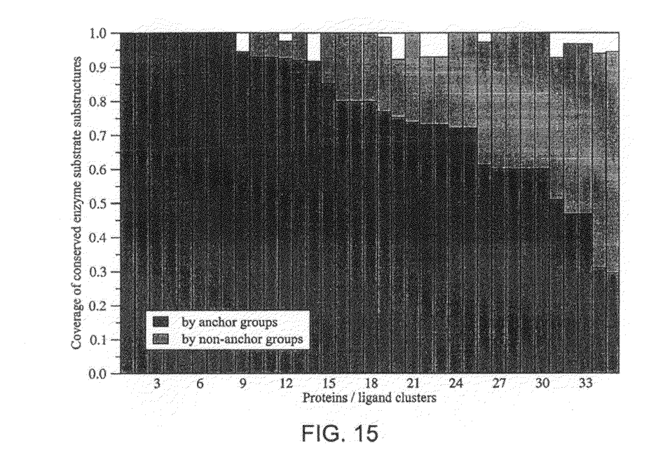

First, from the dataset of 711 protein-ligand complexes, enzymes were selected in which the anchor substructure (or multiple anchor substructures) derived for the top-ranked predicted binding pockets consists of ≧50% and ≦90% of the average ligand molecule's size and matches the native ligand. Subsequently, native ligands were scanned for the presence of CSSs. Here, the collection of the CSSs compiled for 42 major enzyme superfamilies by Babbitt and colleagues (Chiang, R. A., A. Sali, and P. C. Babbitt, Evolutionarily conserved substrate substructures for automated annotation of enzyme superfamilies. PLoS Comput Biol, 2008. 4(8): p. e1000142.) were used, from which were removed those substructures that consisted of less than 5 atoms. A CSS was considered to be present in the native ligand if the native ligand atoms covered at least 90% of its atoms, as reported by SIMCOMP (Hattori, M., et al., Heuristics for chemical compound matching. Genome Inform, 2003. 14: p. 144-53.). This procedure resulted in 24 enzymes and 35 ligand clusters. Next, for each cluster and the associated anchor substructure, the fraction of CSS's atoms covered by the anchor functional groups as well as the fraction covered by the non-anchor substructures was examined. The results presented in FIG. 15 show that the highly conserved substructures of the enzyme substrates identified by Babbitt and colleagues to a large extent overlap with the anchor substructures detected by the threading-based approach; in over 70% of the cases, the anchor substructure covers at least 70% of CSS's atoms. Detailed results obtained for 4-a-glucanotransferase from T. litoralis (PDB-ID: 1k1w) and D-xylose isomerase from Arthrobacter sp. (PDB-ID: 1die) are presented in Tables 5-6.

The structural and chemical patterns of enzyme substrates, or small ligands in general, have been conserved during evolution and maintained by the strong conservation of structural and chemical features of the binding site residues. Importantly, using threading techniques, strongly conserved patterns can be detected in ligands bound to evolutionarily distant proteins. In addition to the identification of pure chemical aspects of the conserved patterns, the approach described here provides consensus binding modes that can be effectively utilized in structure-based modeling, as demonstrated by FINDSITE^{LHM}.

TABLE 5
Multiple common anchor substructures (dashed circles) identified from weakly homologous threading templates for 4-α-glucanotransferase
from T. litoralis (PDB-ID: 1k1w) compared to the conserved substrate substructure reported by Chiang et al. 2008 (solid circles).
The anchor substructures are presented for selected ligand clusters obtained for top-ranked binding pockets.

| | | | | TM-score/ | | |
|---|---|---|---|---|---|---|
| | Ligands | PDB-ID | SID* | RMSD^{†} | SCOP superfamily/family | EC^{‡} |
| Target protein | 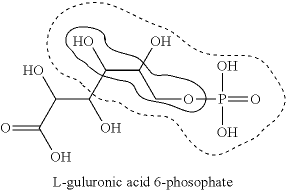 | 1k1w | — | — | Ribulose-phosphate binding barrel/Decarboxylase | 4.1.2.— |
| Cluster: 1 Templates: 45 RMSD^{§}: 2.90 Å | 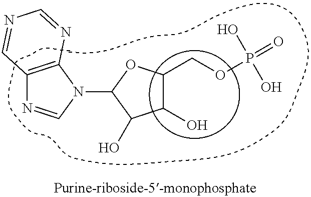 | 1b3o | 19.3% | 0.54/3.18 Å | Inosine monophosphate dehydrogenase (IMPDH)/ Inosine monophosphate dehydrogenase (IMPDH) | 1.1.1.205 |
| | 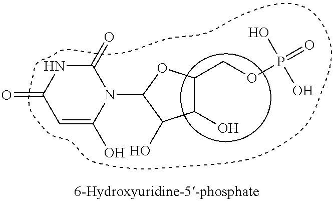 | 1dqx | 19.6% | 0.62/2.50 Å | Ribulose-phosphate binding barrel/Decarboxylase | 4.1.1.23 |
| | 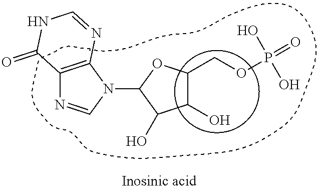 | 1jr1 | 15.8% | 0.41/3.50 Å | Inosine monophosphate dehydrogenase (IMPDH)/ Inosine monophosphate dehydrogenase (IMPDH) | 1.1.1.205 |
| | 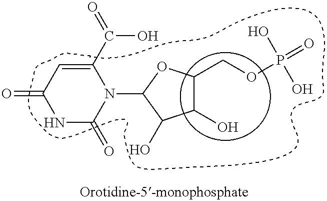 | 1km6 | 24.8% | 0.82/2.12 Å | Ribulose-phosphate binding barrel/Decarboxylase | 4.1.1.23 |
| | 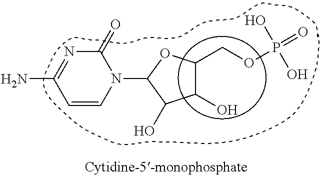 | 1lp6 | 23.3% | 0.82/1.94 Å | Ribulose-phosphate binding barrel/Decarboxylase | 4.1.1.23 |
| | 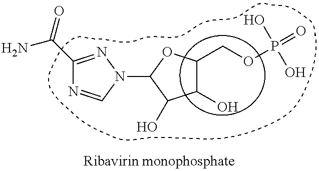 | 1me7 | 15.1% | 0.47/3.56 Å | Inosine monophosphate dehydrogenase (IMPDH)/ Inosine monophosphate dehydrogenase (IMPDH) | 1.1.1.205 |
| | 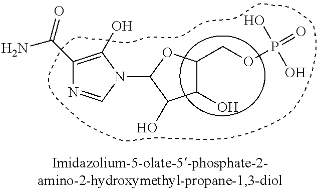 | 1mwf | 15.2% | 0.47/3.55 Å | Inosine monophosphate dehydrogenase (IMPDH)/ Inosine monophosphate dehydrogenase (IMPDH) | 1.1.1.205 |
| | 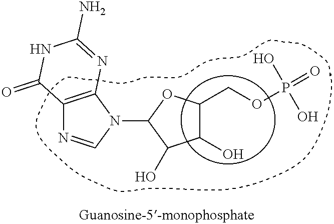 | 2ble | 17.0% | 0.49/3.51 Å | — | 1.7.1.7 |
| | 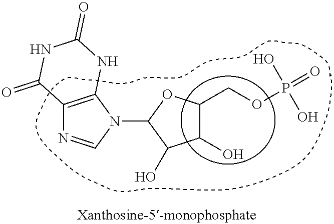 | 2czf | 25.1% | 0.82/2.20 Å | Ribulose-phosphate binding barrel/Decarboxylase | 4.1.1.23 |
| | 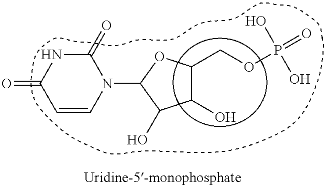 | 2ffc | 18.1% | 0.56/2.65 Å | Ribulose-phosphate binding barrel/Decarboxylase | 4.1.3.23 |
| Cluster: 2 Templates: 20 RMSD^{§}: 3.45 Å | 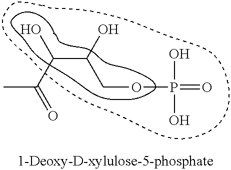 | 1ixn | 20.4% | 0.71/2.91 Å | Pyridoxine 5'-phosphate synthase/Pyridoxine 5'- phosphate synthase | — |
| | 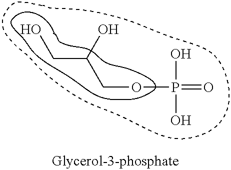 | 1ixo | 20.5% | 0.73/2.92 Å | Pyridoxine 5'-phosphate synthase/Pyridoxine 5'- phosphate synthase | — |
| | 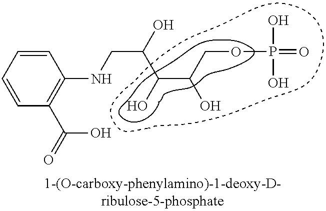 | 1lbm | 15.5% | 0.77/2.96 Å | Ribulose-phosphate binding barrel/Tryptophan biosynthesis enzymes | 5.3.1.24 |
| | 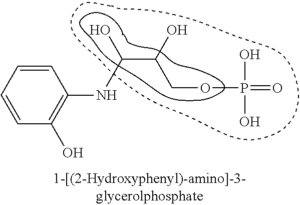 | 1tjp | 16.8% | 0.62/3.49 Å | Ribulose-phosphate binding barrel/Tryptophan biosynthesis enzymes | 4.2.1.20 |
| | 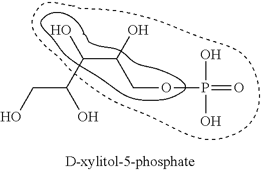 | 2fli | 17.7% | 0.79/2.95 Å | Ribulose-phosphate binding barrel/D-ribulose-5-phosphate 3- epimerase | 5.1.3.— |
| Cluster: 3 Templates: 40 RMSD^{§}: 3.83 Å | 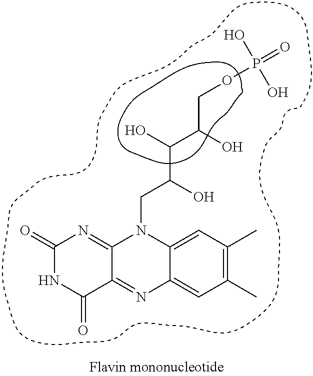 | 1dor | 17.5% | 0.54/3.28 Å | FMN-linked oxidoreductases/ FMN-linked oxidoreductases | 1.3.3.1 |

*Sequence identity.
^{†}TM-score and Cα RMSD of the aligned region reported by TM-Align (Zhang, Y. and J. Skolnick, TM-align: a protein structure alignment algorithm based on the TM-score. Nucleic Acids Res, 2005. 33(7): p. 2302-9.).
^{‡}Enzyme Commission nomenclature.
^{§}Average pairwise RMSD of the anchor heavy atoms calculated for all ligands that belong to a particular cluster.

TABLE 6
Multiple common anchor substructures (solid line) identified from weakly homologous threading templates for D-xylose isomerase from
Arthrobacter sp. (PDB-ID: 1die) compared to the conserved substrate substructure reported by Chiang et al. (dashed line).
The anchor substructures are presented for selected ligand clusters obtained for top-ranked binding pockets.

| | | | | TM-score | | |
|---|---|---|---|---|---|---|
| | Ligands | PDB-ID | SID* | RMSD^{†} | SCOP superfamily/family | EC^{‡} |
| Target protein | 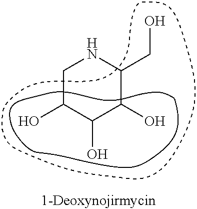 | 1die | — | — | Xylose isomerase-like/ Xylose isomerase | 5.3.1.5 |
| Cluster: 1 Templates: 12 RMSD^{§}: 3.49 Å | 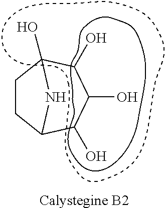 | 2cbv | 19.5% | 0.53/4.65 Å | (Trans)glycosidases/ Family 1 of glycosyl hydrolase | 3.2.1.21 |
| | 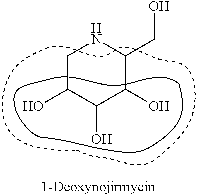 | 1oim | 19.5% | 0.53/4.64 Å | (Trans)glycosidases/ Family 1 of glycosyl hydrolase | 3.2.1.21 |
| | 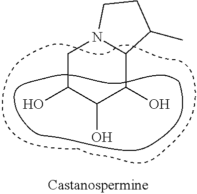 | 2cbu | 19.7% | 0.54/4.67 Å | (Trans)glycosidases/ Family 1 of glycosyl hydrolase | 3.2.1.21 |
| | 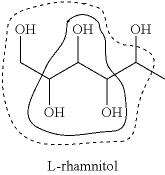 | 1de5 | 19.3% | 0.68/3.45 Å | Xylose isomerase-like/ L-rhamnose isomerase | 5.3.1.14 |
| | 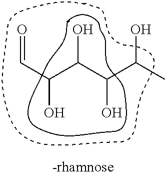 | 1de6 | 19.3% | 0.68/3.50 Å | Xylose isomerase-like/ L-rhamnose isomerase | 5.3.1.14 |
| | 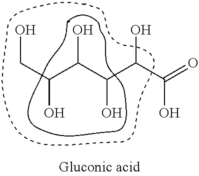 | 1bgg | 18.8% | 0.53/4.66 Å | (Trans)glycosidases/ Family 1 of glycosyl hydrolase | 3.2.1.21 |
| | 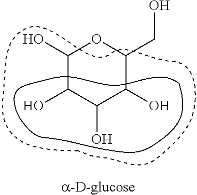 | 1hiz | 16.4% | 0.55/5.05 Å | (Trans)glycosidases/ Beta-glycanases | 3.2.1.8 |
| | 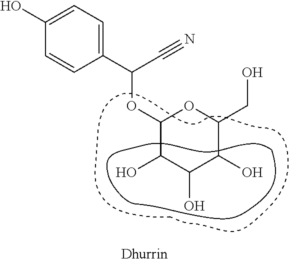 | 1e55 | 20.1% | 0.49/4.69 Å | (Trans)glycosidases/ Family 1 of glycosyl hydrolase | 3.2.1.21 |
| Cluster: 2 Templates: 12 RMSD^{§}: 5.61 Å | 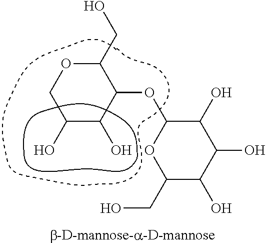 | 2man | 15.3% | 0.63/4.38 Å | (Trans)glycosidases/ Beta-glycanases | 3.2.1.78 |
| | 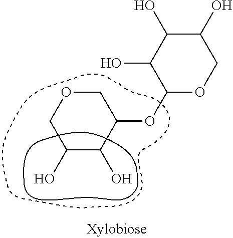 | 1vbr | 17.9% | 0.62/4.89 Å | (Trans)glycosidases/ Beta-glycanases | 3.2.1.8 |
| | 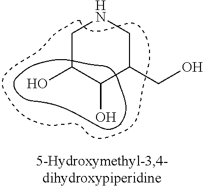 | 1oif | 19.8% | 0.53/4.61 Å | (Trans)glycosidases/ Family 1 of glycosyl hydrolase | 3.2.1.21 |
| | 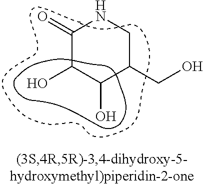 | 1uz4 | 19.2% | 0.54/4.75 Å | (Trans)glycosidases/ Beta-glycanases | — |
| | 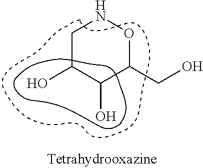 | 1w3j | 19.3% | 0.53/4.62 Å | (Trans)glycosidases/ Beta-glycanases | 3.2.1.21 |
| | 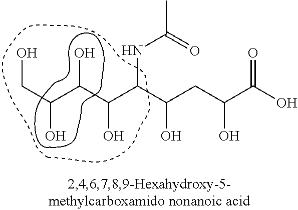 | 1f73 | 17.5% | 0.59/3.92 Å | Aldolase/Class I aldolase | 4.1.3.3 |
| | 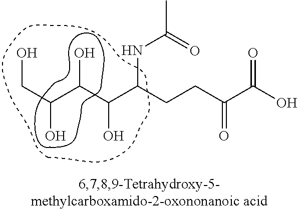 | 1f74 | 17.5% | 0.58/4.11 Å | Aldolase/Class I aldolase | 4.1.3.3 |
| | 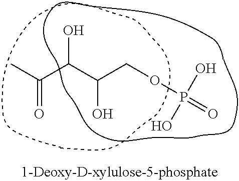 | 1ixn | 19.6% | 0.69/3.76 Å | Pyridoxine 5′-phosphate synthase/ Pyridoxine 5′-phosphate synthase | — |
| Cluster: 3 Templates: 12 RMSD: 3.86 Å | 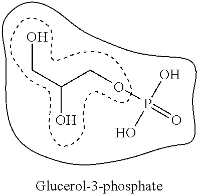 | 1ixo | 19.3% | 0.71/3.78 Å | Pyridoxine 5′-phosphate synthase/ Pyridoxine 5′-phosphate synthase | — |
| | 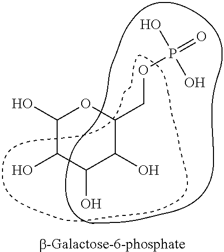 | 4pbg | 21.2% | 0.51/4.49 Å | Trans)glycosidases/ Family 1 of glycosyl hydrolase | 3.2.1.85 |
| Cluster: 4 Templates: 2 RMSD: 0.71 Å | 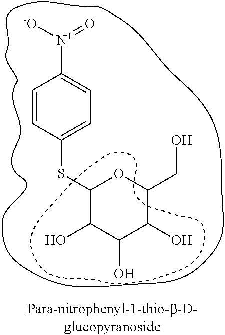 | 1elf | 19.5% | 0.49/4.67 Å | (Trans)glycosidases/ Family 1 of glycosyl hydrolase | 3.2.1.21 |

*Sequence identity.
^{†}TM-score and Cα RMSD of the aligned region reported by TM-Align (Zhang, Y. and J. Skolnick, TM-align: a protein structure alignment algorithm based on the TM-score. Nucleic Acids Res, 2005. 33(7): p. 2302-9.).
^{‡}Enzyme Commission nomenclature.
^{§}Average pairwise RMSD of the anchor heavy atoms calculated for all ligands that belong to a particular cluster.

Conservation of protein sequence and structural patterns has been used previously to study protein molecular function. Sequence entropy analysis suggests that residues contacting anchor functional groups have been subjected to higher evolutionary conservation than those contacting ligand variable regions. Furthermore, the conservation of the anchor-binding pose is consistent with the relatively low experimental B-factors observed for residues contacting anchor functional groups. The significantly higher structural plasticity of the variable region binding residues reflects different types and sizes of functional groups found in the ligand variable substructures that might be responsible for ligand specificity for particular protein family members. Binding site characteristics are important for understanding how binding sites maintain selectivity for particular ligands and predicting ligand cross-reactivity.

In addition, detailed information about bioactive molecules, such as the approximate orientation in the complexed state, the extent of chemical conservation, the complementary features of the interacting protein residues, and the like, can be explicitly incorporated into the ligand-binding site modeling. Using the ligand binding modes from related structures incorporated directly as spatial restraints in protein structure modeling by MOBILE was shown to provide realistic homology models of protein binding sites. This idea is in fact more general and applies to evolutionarily distant proteins as well. Evolution provides a type of signal averaging to identify the essential features associated with ligand binding. This insight can be profitably exploited in a variety of contexts.

In large-scale computational experiments involving ligand docking, the conditional transfer of ligands from known structures of related protein-ligand complexes can be an effective alternative to CPU-expensive, classical ligand docking approaches. FINDSITE^{LHM} was developed based in part on the observation that across a set of weakly related proteins, not only is the chemical identity of anchor functional groups strongly conserved, but also the anchor binding mode, with an average pairwise RMSD <2.5 Å in most of the cases. FINDSITE^{LHM} directly uses the consensus binding mode of an anchor substructure as the reference coordinates to perform rapid flexible ligand docking by superposition. Particularly for predicted protein structures, FINDSITE^{LHM} can outperform all-atom ligand docking approaches in terms of the fraction of recovered binding residues and specific native contacts and requires considerably less CPU time. Since for the majority of gene products, homologous and weakly homologous proteins can be identified in structural databases by current threading methods and approximately correct protein models can be generated by protein structure prediction techniques, the results obtained by FINDSITE^{LHM} offer the possibility of proteome-scale structure-based virtual screening for novel biopharmaceutical discovery. Virtual screening at the proteome level has a great advantage over the screening of single proteins. It affords the differential analysis of putative bioactive molecules selected for multiple drug targets to identify ligands unique to a single protein family. These molecules might represent lead compounds with desired selectivity that could be further exploited at the outset of a drug development process to reduce side effects.

A similar methodology is implemented in the AnnoLyze program (Marti-Renom, M. A., et al., The AnnoLite and AnnoLyze programs for comparative annotation of protein structures. BMC Bioinformatics, 2007. 8 Suppl 4: p. S4.) that transfers small ligands and interacting domains from homologous structures. With the required by AnnoLyze minimum of 20% sequence identity and structural alignments of high quality and coverage, known ligands from the LigBase can be accurately transferred into the target proteins. Here, the analysis was generalized to evolutionarily far more distant proteins and the subset of the ligands whose pose is conserved, viz. the anchor region was identified. Furthermore, similarity in global fold can be insufficient for effective function interference and results in a high false positive rate. For that reason, the most effective function prediction methods, such as ProFunc (Laskowski, R. A., J. D. Watson, and J. M. Thornton, ProFunc: a server for predicting protein function from 3D structure. Nucleic Acids Res, 2005. 33(Web Server issue): p. W89-93.), AnnoLite (Marti-Renom, M. A., et al., The AnnoLite and AnnoLyze programs for comparative annotation of protein structures. BMC Bioinformatics, 2007. 8 Suppl 4: p. S4.), or Mark-Us (Nayal, M., B. C. Hitz, and B. Honig, GRASS: a server for the graphical representation and analysis of structures. Protein Sci, 1999. 8(3): p. 676-9.) typically combine structure- and sequence-based techniques. An important component of FINDSITE/FINDSITE^{LHM} is the template selection by threading that employs a strong sequence profile factor. Sensitive protein threading detects evolutionarily distant homologues to provide a set of templates with clear functional relationships to the protein of interest not only in terms of the localization of the binding site but also the detailed chemical and structural aspects of ligand binding, particularly those that impact binding specificity. Thus, threading can provide a richness to functional annotation that to date was not fully exploited.

### Example 2

### Binding Site Prediction

In this example, the structures of protein-ligand complexes used were selected from the Protein Data Bank (PDB). First, ligand-bound forms were identified, where noncovalently bound organic molecules, cofactors, nucleotides, and short peptides composed of standard or modified amino acids were considered as ligands if the number of atoms was greater than 6 and less than 100. Proteins having more than one ligand in the binding pocket were removed. Because proteins consisting of greater than 400 amino acid residues cannot be modeled using TASSER (Zhang, Y, A. K. Arakaki, and J. Skolnick, TASSER: an automated method for the prediction of protein tertiary structures in CASP6. Proteins, 2005. 61 Suppl 7: p. 91-8.) in a reasonable amount of computer time, these proteins were excluded. No two proteins in the dataset share greater than 35% sequence identity. This restriction was chosen for benchmarking purposes only. Non-benchmarking applications may not utilize this restriction. The resulting set consisted of 901 protein-ligand complexes.

In this example, the ligand-binding information for the non-redundant set of 901 proteins was predicted and the results were compared to experimental data. In this experiment only threading templates that are weakly homologous (e.g., less than 35% sequence identity) to their targets were considered. For crystal structures used in the ligand-binding site prediction, considering a cutoff distance of 4 Å as the hit criterion, the success rate was 70.9% for identifying the best of top five predicted ligand-binding sites with the corresponding ranking accuracy of 76.0%. High prediction accuracy as well as the ability to correctly rank identified binding sites was sustained when low-to-moderate quality protein models were used instead of experimental structures, showing a 67.3% success rate with 75.5% ranking accuracy.

Overall accuracy of binding site prediction correlated with the estimated confidence level: the average distance between the centers of predicted and observed binding pockets calculated for top-ranked solutions was ˜2 Å, ˜5 Å and ˜10 Å for Easy, Medium and Hard targets, respectively. Median specificity and sensitivity of predicting ligand-binding residues was 0.96 and 0.64, respectively. This corresponds to the accuracy and the Matthew's correlation coefficient between predicted and observed binding residues of 0.93 and 0.59, respectively. The average number of predicted binding residues (20.0±7.3 and 20.3±6.8 for the best and the top identified pockets, respectively) was substantially in agreement with that observed in the crystal structures of protein-ligand complexes (19.6±6.5).

Ligand-based virtual screening against the KEGG compound library (Kanehisa, M. and S. Goto, KEGG: kyoto encyclopedia of genes and genomes. Nucleic Acids Res, 2000. 28(1): p. 27-30.) (release 44.0+/10-15, Oct. 07) using predicted ligand templates as multiple query compounds resulted in the enrichment factor better than random in 78% of the cases for accurately predicted binding sites (whose center of mass is within 4 Å of the experimental one) and in 38% of the cases for less accurately predicted binding pockets (distance >4 Å).

Molecular function was transferred from threading-templates to target proteins with a precision and sensitivity (recall) of 0.76 and 0.54, respectively, considering all 7825 molecular function terms provided by the Gene Ontology. Many individual molecular functions are accurately transferable; these cover a broad spectrum of molecular events including both enzymatic and binding activities.

### Example 3

### Binding Site Validation on a True Negative Set

In this example, a dataset that consisted of 281 protein-protein dimeric interfaces was formed by M-TASSER Chen, H. and J. Skolnick, M-TASSER: an algorithm for protein quaternary structure prediction. Biophys J, 2008. 94(3): p. 918-28) using 562 non-redundant protein chains. This dataset was used as a negative set, with the assumption that interfacial residues involving direct interactions between proteins should not bind small molecule ligands. For this example, a protein residue was considered as binding a ligand if any of its heavy atoms lie within a distance of 4 Å from the predicted binding site center. For the top predicted binding pockets, more than 5% interface residues were misclassified as belonging to a ligand-binding pocket in 8.4% of the cases.

### Example 4

### Virtual Ligand Docking

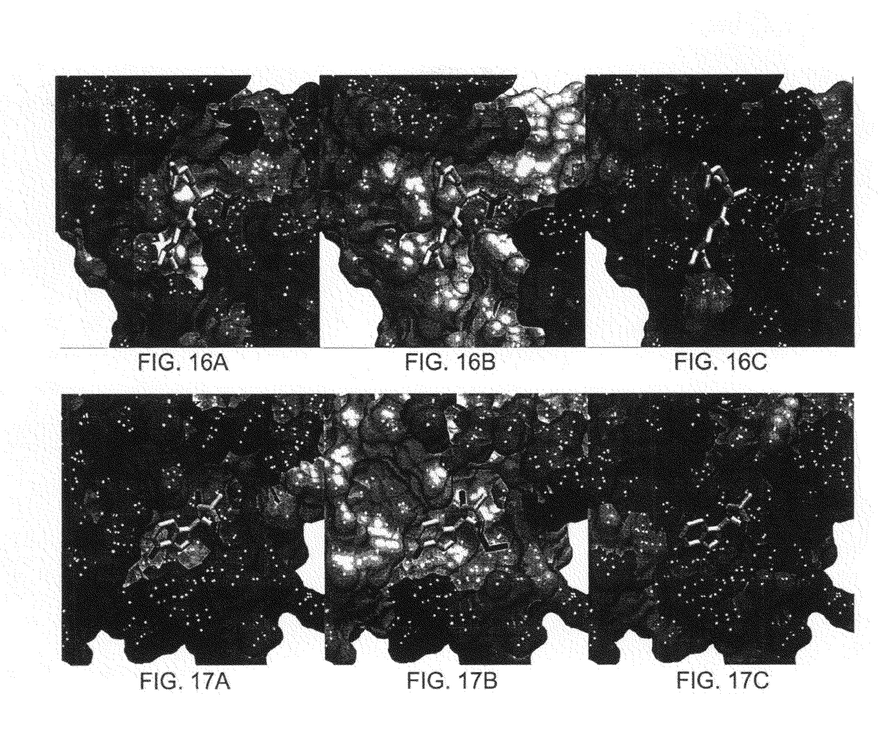

In this example, results of the application of FINDSITE^{LHM} to glutathione S-transferase (PDB-ID: 1a0f), MTA phosphorylase (PDB-ID: 1sd2) and lysine aminotransferase (PDB-ID: 2cjd) is described. Tables 7-9 illustrate common anchor substructures identified from weakly homologous threading templates as well as different variable groups found in ligands complexed with the template proteins. Referring now to FIGS. 16-18, the degree of sequence and structure conservation of amino acid residues for these proteins is projected onto the target protein surface and compared to a random distribution.

Referring now to FIGS. 16A-C, FIG. 16A depicts the sequence entropy for the binding site of glutathione sulfonic acid complexed with glutathione S-transferase (PDB-ID: 1a0f), where light gray protein indicates low sequence entropy and dark gray protein indicates high sequence entropy. The anchor part of the bound molecule is shown in white, whereas the variable part is shown in black. FIG. 16B depicts the normalized crystallographic B-factors where light gray protein indicates low B-factors and dark gray protein indicates high B-factors. FIG. 16C depicts random values (light gray—0.0, light gray—1.0) for the binding site.

Referring now to FIGS. 17A-C, FIG. 17A depicts the sequence entropy for the binding site of 5′-methylthiotubercidin complexed with MTA phosphorylase (PDB-ID: 1sd2), where light gray protein indicates low sequence entropy and dark gray protein indicates high sequence entropy. The anchor part of the bound molecule is shown in white, whereas the variable part is shown in black. FIG. 17B depicts the normalized crystallographic B-factors where light gray protein indicates low B-factors and dark gray protein indicates high B-factors. FIG. 17C depicts random values (light gray—0.0, light gray—1.0) for the binding site.

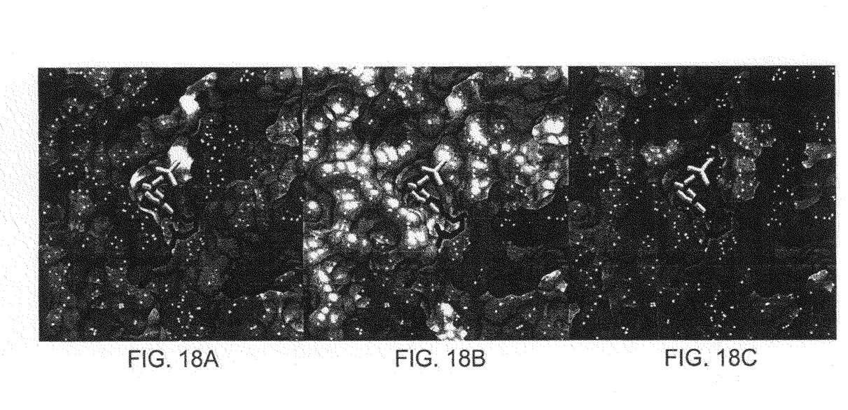

Referring now to FIGS. 18A-C, FIG. 18A depicts the sequence entropy for the binding site of lysine and piridoxal-5′-phosphate complexed with lysine aminotransferase (PDB-ID: 2cjd), where light gray protein indicates low sequence entropy and dark gray protein indicates high sequence entropy. The anchor part of the bound molecule is shown in white, whereas the variable part is shown in black. FIG. 18B depicts the normalized crystallographic B-factors where light gray protein indicates low B-factors and dark gray protein indicates high B-factors. FIG. 18C depicts random values (light gray—0.0, light gray—1.0) for the binding site.

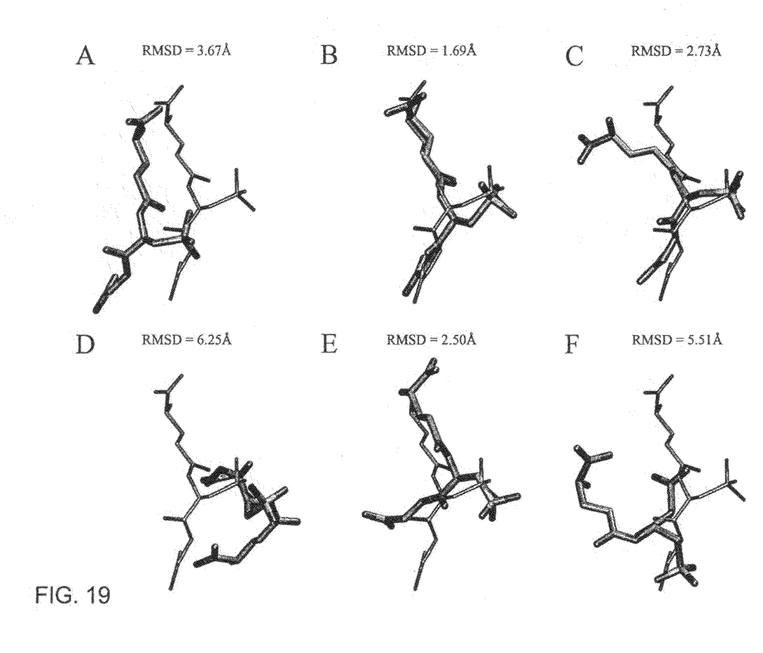

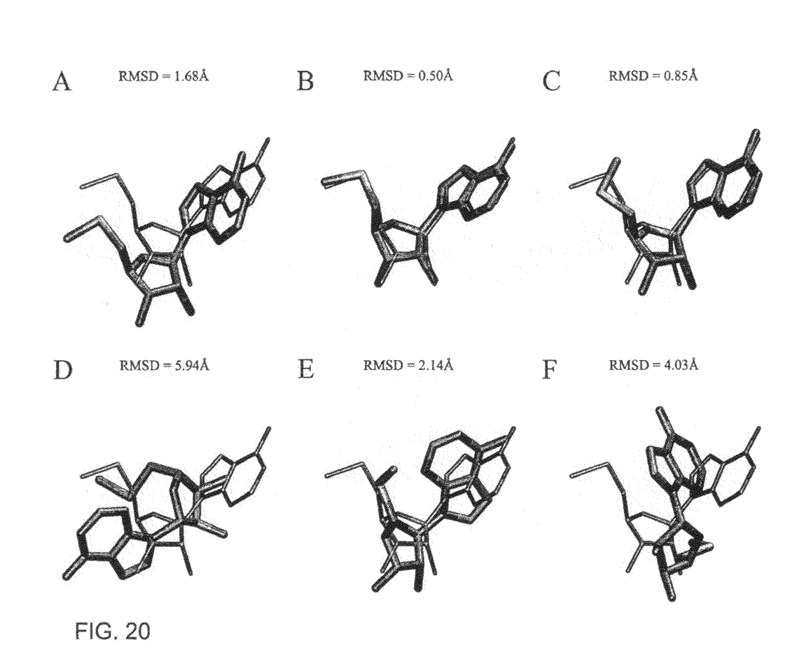

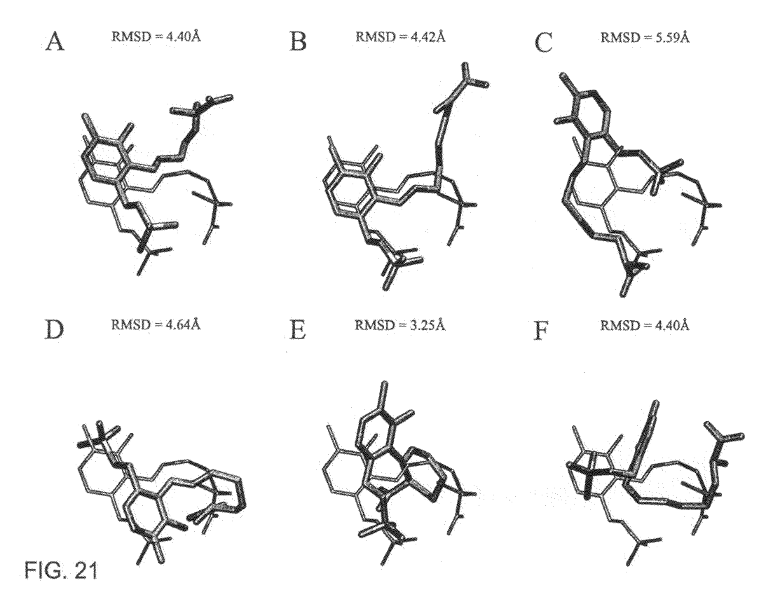

As expected, residues in contact with anchor regions are more strongly conserved and have lower B-factors. Referring now to FIGS. 19-21, the results of flexible ligand docking by FINDSITE^{LHM} (including the post-docking refinement) are compared to ligand binding poses predicted by classical docking approaches and the consistently better performance of FINDSITE^{LHM} is demonstrated.

TABLE 7
Common anchor substructure (A) identified from weakly homologous threading templates for glutathione S-transferase from E.coli
(PDB-ID: 1a0f) as well as different variable groups (R) found in ligands complexed with the template proteins.

| | | | | TM- | | |
|---|---|---|---|---|---|---|
| | | | | score/ | SCOP | |
| Anchor (A) | Variable part (R) | PDB ID | SID* | RMSD^{†} | superfamily/family | EC^{‡} |
| 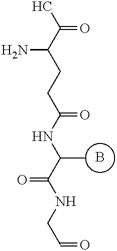 | 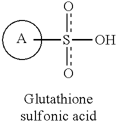 | 1a0f(target) | — | — | Thioredoxin-like/Glutathione S-transferase, N-terminal domain Glutathione S-transferase, C- terminal domain/Glutathione S-transferase, C-terminal domain | 2.5.1.18 |
| | 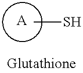 | 19gs | 19.1% | 0.80/ 2.65 Å | Thioredoxin-like/Glutathione S-transferase, N-terminal domain Glutathione S-transferase, C- terminal domain/Glutathione S-transferase, C-terminal domain | 2.5.1.18 |
| | 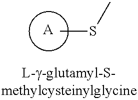 | 2ab6 | 24.1% | 0.81/ 2.47 Å | Thioredoxin-like/Glutathione S-transferase, N-terminal domain Glutathione S-transferase, C- terminal domain/Glutathione S-transferase, C-terminal domain | 2.5.1.18 |
| | 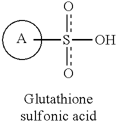 | 1ev4 | 21.1% | 0.77/ 2.80 Å | Thioredoxin-like/Glutathione S-transferase, N-terminal domain Glutathione S-transferase, C- terminal domain/Glutathione S-transferase, C-terminal domain | 2.5.1.18 |
| | 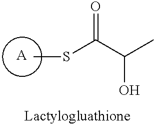 | 1axd | 26.3% | 0.80/ 2.59 Å | Thioredoxin-like/Glutathione S-transferase, N-terminal domain Glutathione S-transferase, C- terminal domain/Glutathione S-transferase, C-terminal domain | 2.5.1.18 |
| | 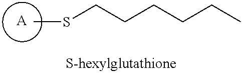 | 17gs | 19.4% | 0.79/ 2.67 Å | Thioredoxin-like/Glutathione S-transferase, N-terminal domain Glutathione S-transferase, C- terminal domain/Glutathione S-transferase, C-terminal domain | 2.5.1.18 |
| | 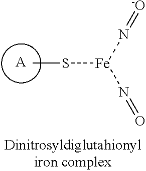 | 1zgn | 18.7% | 0.80/ 2.65 Å | Thioredoxin-like/Glutathione S-transferase, N-terminal domain Glutathione S-transferase, C- terminal domain/Glutathione S-transferase, C-terminal domain | 2.5.1.18 |
| | 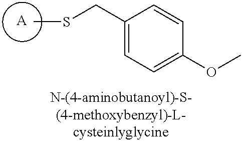 | 1pl1 | 19.4% | 0.76/ 2.78 Å | Thioredoxin-like/Glutathione S-transferase, N-terminal domain Glutathione S-transferase, C- terminal domain/Glutathione S-transferase, C-terminal domain | 2.5.1.18 |
| | 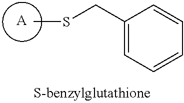 | 1guh | 19.4% | 0.76/ 2.81 Å | Thioredoxin-like/Glutathione S-transferase, N-terminal domain Glutathione S-transferase, C- terminal domain/Glutathione S-transferase, C-terminal domain | 2.5.1.18 |
| | 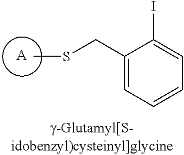 | 1m9b | 17.1% | 0.80/ 2.50 Å | Thioredoxin-like/Glutathione S-transferase, N-terminal domain Glutathione S-transferase, C- terminal domain/Glutathione S-transferase, C-terminal domain | 2.5.1.18 |
| | 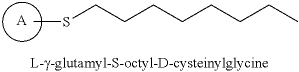 | 1u88 | 18.1% | 0.81/ 2.51 Å | | 2.5.1.18 |
| | 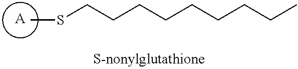 | 12gs | 18.7% | 0.80/ 2.63 Å | Thioredoxin-like/Glutathione S-transferase, N-terminal domain Glutathione S-transferase, C- terminal domain/Glutathione S-transferase, C-terminal domain | 2.5.1.18 |
| | 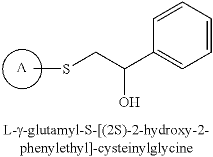 | 2c4j | 24.1% | 0.81/ 2.39 Å | Thioredoxin-like/Glutathione S-transferase, N-terminal domain Glutathione S-transferase, C- terminal domain/Glutathione S-transferase, C-terminal domain | 2.5.1.18 |
| | 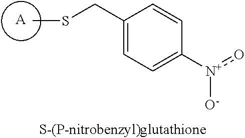 | 1glq | 20.2% | 0.79/ 2.69 Å | Thioredoxin-like/Glutathione S-transferase, N-terminal domain Glutathione S-transferase, C- terminal domain/Glutathione S-transferase, C-terminal domain | 2.5.1.18 |
| | 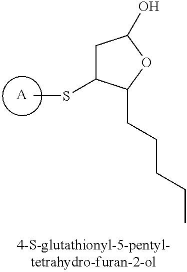 | 1b48 | 23.5% | 0.74/ 3.02 Å | Thioredoxin-like/Glutathione S-transferase, N-terminal domain Glutathione S-transferase, C- terminal domain/Glutathione S-transferase, C-terminal domain | 2.5.1.18 |
| | 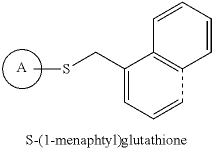 | 3ljr | 23.8% | 0.84/ 2.60 Å | Thioredoxin-like/Glutathione S-transferase, N-terminal domain Glutathione S-transferase, C- terminal domain/Glutathione S-transferase, C-terminal domain | 2.5.1.18 |
| | 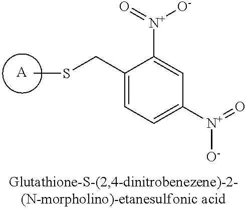 | 18gs | 18.7% | 0.80/ 2.63 Å | Thioredoxin-like/Glutathione S-transferase, N-terminal domain Glutathione S-transferase, C- terminal domain/Glutathione S-transferase, C-terminal domain | 2.5.1.18 |
| | 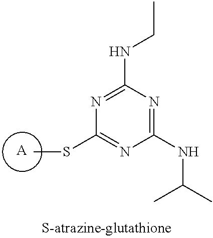 | 1bye | 26.1% | 0.81/ 2.55 Å | Thioredoxin-like/Glutathione S-transferase, N-terminal domain Glutathione S-transferase, C- terminal domain/Glutathione S-transferase, C-terminal domain | |
| | 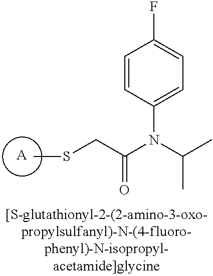 | 1bx9 | 25.7% | 0.78/ 2.84 Å | Thioredoxin-like/Glutathione S-transferase, N-terminal domain Glutathione S-transferase, C- terminal domain/Glutathione S-transferase, C-terminal domain | |
| | 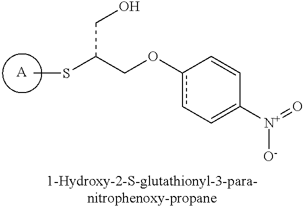 | 1c72 | 21.4% | 0.80/ 2.61 Å | Thioredoxin-like/Glutathione S-transferase, N-terminal domain Glutathione S-transferase, C- terminal domain/Glutathione S-transferase, C-terminal domain | 2.5.1.18 |
| | 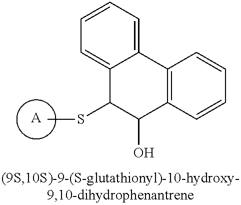 | 1b4p | 23.6% | 0.81/ 2.52 Å | Thioredoxin-like/Glutathione S-transferase, N-terminal domain Glutathione S-transferase, C- terminal domain/Glutathione S-transferase, C-terminal domain | 2.5.1.18 |
| | 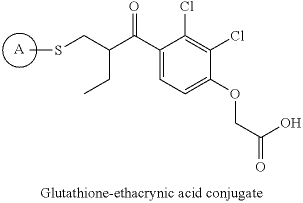 | 1lgs | 18.7% | 0.80/ 2.62 Å | Thioredoxin-like/Glutathione S-transferase, N-terminal domain Glutathione S-transferase, C- terminal domain/Glutathione S-transferase, C-terminal domain | 2.5.1.18 |
| | 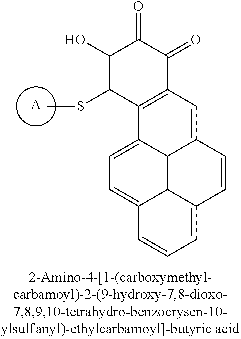 | 1f3b | 20.4% | 0.77/ 2.70 Å | Thioredoxin-like/Glutathione S-transferase, N-terminal domain Glutathione S-transferase, C- terminal domain/Glutathione S-transferase, C-terminal domain | 2.5.1.18 |

*Sequence identity.
^{†}TM-score and Cα RMSD of the aligned region reported by TM-Align (Zhang, Y. and J. Skolnick, TM-align: a protein structure alignment algorithm based on the TM-score. Nucleic Acids Res, 2005. 33(7): p. 2302-9.).
^{‡}Enzyme Commission nomenclature.

TABLE 8
Common anchor substructure (A) identified from weakly homologous threading templates for the human MTA phosphorylase (PDB-ID: 1sd2;
SCOP superfamily/family: Purine and uridine phosphorylases/Purine and uridine phosphorylases; EC: 2.4.2.28)
as well as different variable groups (at the positions R₁-R₇) found in ligands complexed with the template proteins.

| 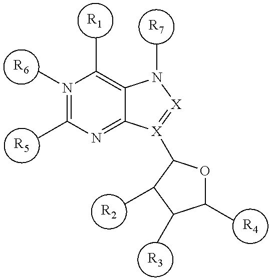 Anchor (A) | | | | | | | | | | |
|---|---|---|---|---|---|---|---|---|---|---|
| | | | | | | | | | TM- | |
| Variable part (R) | | | | | | | PDB | | score/ | |
| R₁ | R₂ | R₃ | R₄ | R₅ | R₆ | R₇ | ID | SID* | RMSD^{†} | EC^{‡} |
| 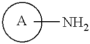 | 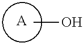 | 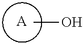 | 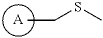 | | | | 1sd2 (tar- get) | — | — | 2.4.2.1 |
| 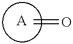 | 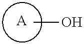 | 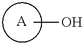 | 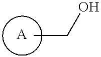 | | | | 1a69 | 20.1% | 0.78/ 3.03 Å | 2.4.2.1 |
| 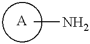 | 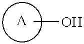 | 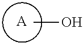 | 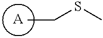 | | | | 1jdt | 19.3% | 0.80/ 2.91 Å | 2.4.2.28 |
| 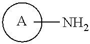 | 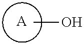 | 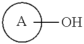 | 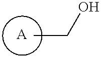 | | | | 1jdv | 19.3% | 0.80/ 2.87 Å | 2.4.2.28 |
| 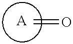 | 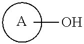 | 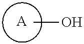 | 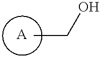 | 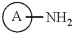 | | | 1je1 | 19.3% | 0.79/ 3.04 Å | 2.4.2.28 |
| 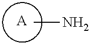 | 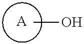 | 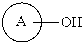 | 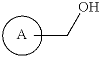 | | 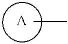 | | 1k9s | 20.1% | 0.77/ 3.04 Å | 2.4.2.1 |
| 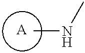 | 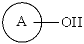 | 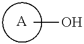 | 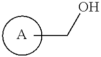 | | | | 1k9s | 20.1% | 0.78/ 2.93 Å | 2.4.2.1 |
| 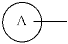 | 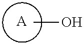 | 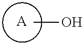 | 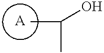 | | | | 1oum | 20.3% | 0.78/ 3.04 Å | 2.4.2.1 |
| 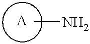 | 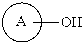 | 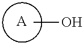 | 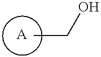 | 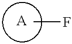 | | | 1pk9 | 20.1% | 0.77/ 3.08 Å | 2.4.2.1 |
| 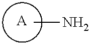 | | 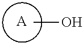 | 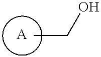 | 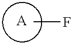 | | | 1pke | 20.2% | 0.77/ 3.09 Å | 2.4.2.1 |
| 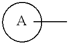 | | 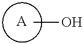 | 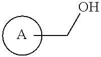 | | | | 1pr2 | 20.1% | 0.77/ 3.12 Å | 2.4.2.1 |
| 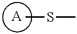 | 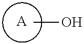 | 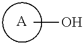 | 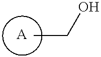 | | | | 1pr4 | 20.2% | 0.78/ 3.06 Å | 2.4.2.1 |
| 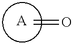 | | | 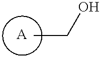 | | | | 1v3q | 23.5% | 0.74/ 2.43 Å | 2.4.2.1 |
| 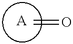 | 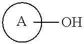 | | 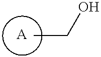 | 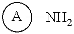 | | | 1v45 | 23.5% | 0.74/ 2.59 Å | 2.4.2.1 |
| 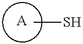 | 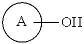 | 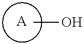 | 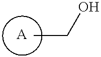 | 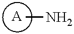 | | 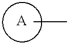 | 1yry | 23.5% | 0.74/ 2.61 Å | 2.4.2.1 |
| 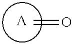 | | 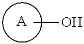 | 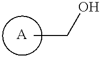 | | | | 1z39 | 15.4% | 0.77/ 3.11 Å | 2.4.2.1 |

*Sequence identity.
^{#}TM-score and Cα RMSD of the aligned region reported by TM-Align (Zhang, Y. and J. Skolnick, TM-align: a protein structure alignment algorithm based on the TM-score. Nucleic Acids Res, 2005. 33(7): p. 2302-9.).
^{‡}Enzyme Commission nomenclature.

TABLE 9
Common anchor substructure (A) identified from weakly homologous threading templates for lysine aminotransferase

| | | | | TM-score/ | SCOP | |
|---|---|---|---|---|---|---|
| Anchor (A) | Variable part (R) | PDB ID | SID* | RMSD^{†} | superfamily/family | EC^{‡} |
| 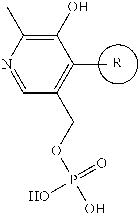 | 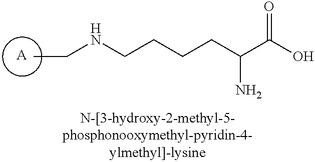 | 1sd2 | — | — | — | — |
| | 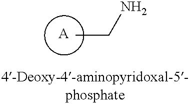 | 1bkg | 21.4% | 0.67/4.47 Å | PLP-dependent transferases/ AAT-like | 2.6.1.1 |
| | 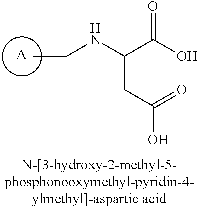 | 1gc4 | 21.2% | 0.67/4.39 Å | PLP-dependent transferases/ AAT-like | 2.6.1.1 |
| | 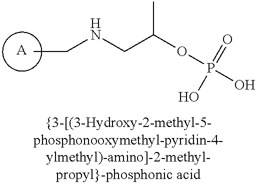 | 1lc8 | 20.5% | 0.74/3.78 Å | PLP-dependent transferases/ AAT-like | |
| | 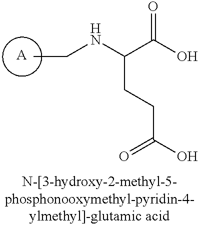 | 1gde | 18.7% | 0.65/4.59 Å | PLP-dependent transferases/ AAT-like | 2.6.1.— |
| | 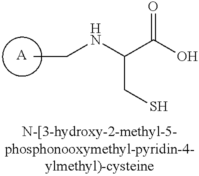 | 1ecx | 16.1% | 0.74/3.90 Å | PLP-dependent transferases/ Cystathione synthase-like | |
| | 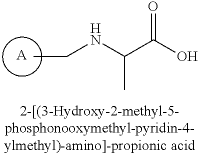 | 1elu | 18.3% | 0.73/3.98 Å | PLP-dependent transferases/ Cystathione synthase-like | |
| | 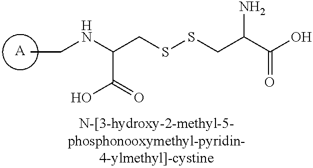 | 1n31 | 18.8% | 0.73/3.97 Å | PLP-dependent transferases/ Cystathione synthase-like | 4.4.1.— |
| | 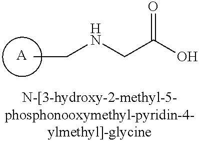 | 1kl1 | 20.1% | 0.71/3.92 Å | PLP-dependent transferases/ GABA- aminotransferase- like | 2.1.2.1 |
| | 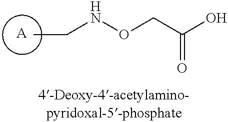 | 1sff | 29.4% | 0.88/2.22 Å | PLP-dependent transferases/ GABA- aminotransferase- like | 2.6.1.19 |
| | 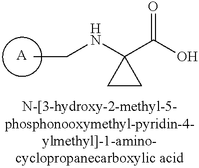 | 1d7r | 25.3% | 0.87/2.50 Å | PLP-dependent transferases/ GABA- aminotransferase- like | 4.1.1.64 |
| | 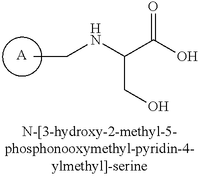 | 1kkp | 20.1% | 0.71/3.94 Å | PLP-dependent transferases/ GABA- aminotransferase- like | 2.1.2.1 |
| | 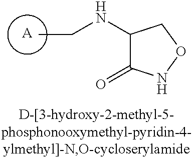 | 1d7s | 25.3% | 0.88/2.49 Å | PLP-dependent transferases/ GABA- aminotransferase- like | 4.1.1.64 |
| | 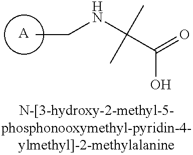 | 1d7v | 25.3% | 0.87/2.51 Å | PLP-dependent transferases/ GABA- aminotransferase- like | 4.1.1.64 |
| | 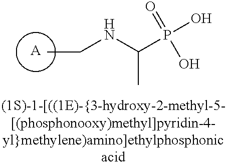 | 1m0q | 25.3% | 0.87/2.48 Å | PLP-dependent transferases/ GABA- aminotransferase- like | 4.1.1.64 |
| | 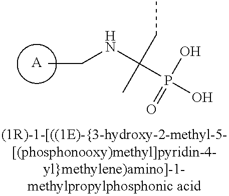 | 1m0o | 25.3% | 0.88/2.49 Å | PLP-dependent transferases/ GABA- aminotransferase- like | 4.1.1.64 |
| | 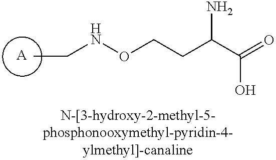 | 2can | 28.0% | 0.89/2.35 Å | PLP-dependent transferases/ GABA- aminotransferase- like | 2.6.1.13 |
| | 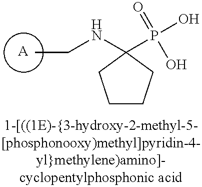 | 1m0n | 25.3% | 0.87/2.52 Å | PLP-dependent transferases/ GABA- aminotransferase- like | 4.1.1.64 |
| | 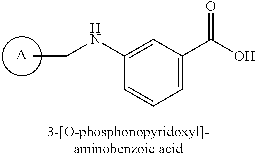 | 1b9i | 21.1% | 0.68/3.62 Å | PLP-dependent transferases/ GABA- aminotransferase- like | |
| | 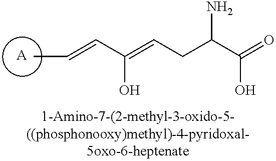 | 2oat | 27.9% | 0.89/2.34 Å | PLP-dependent transferases/ GABA- aminotransferase- like | 2.6.1.13 |
| | 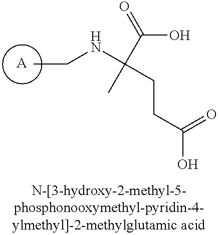 | 1bjo | 16.7% | 0.73/4.23 Å | PLP-dependent transferases/ GABA- aminotransferase- like | 2.6.1.52 |
| | 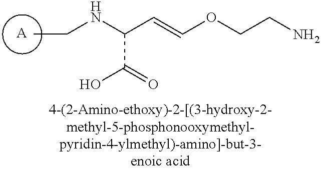 | 1m7y | 15.8% | 0.63/4.95 Å | PLP-dependent transferases/ GABA- aminotransferase- like | 4.4.1.14 |
| | 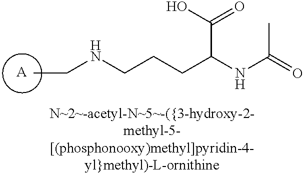 | 1wkg | 29.7% | 0.92/1.98 Å | PLP-dependent transferases/ GABA- aminotransferase- like | 2.6.1.11 |
| | 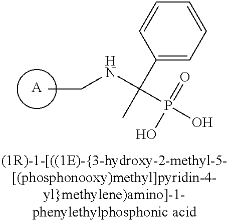 | 1m0p | 25.3% | 0.87/2.51 Å | PLP-dependent transferases/ GABA- aminotransferase- like | 4.1.1.64 |
| | 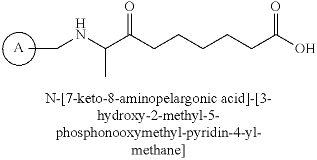 | 1dj9 | 20.6% | 0.77/3.37 Å | PLP-dependent transferases/ GABA- aminotransferase- like | 2.3.1.47 |
| | 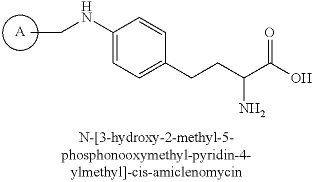 | 1mly | 24.9% | 0.86/2.71 Å | PLP-dependent transferases/ GABA- aminotransferase- like | 2.6.1.62 |
| | 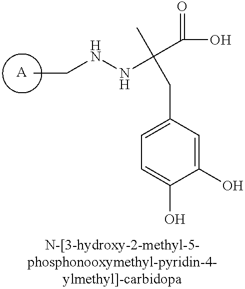 | 1js3 | 19.8% | 0.66/4.06 Å | PLP-dependent transferases/ Pyridoxal- dependent decarboxylase | 4.1.1.28 |

*Sequence identity.
^{†}TM-score and Cα RMSD of the aligned region reported by TM-Align (Zhang, Y. and J. Skolnick, TM-align: a protein structure alignment algorithm based on the TM-score. Nucleic Acids Res, 2005. 33(7): p. 2302-9.).
^{‡}Enzyme Commission nomenclature.

### Example 5

### Ligand-Based Virtual Screening

In this example, the ligand-based virtual screening methodology was applied to identify HIV-1 protease inhibitors. HIV-1 protease is an aspartic protease that cleaves the nascent polyproteins during viral replication. The important role of HIV-1 protease in the life cycle of HIV has motivated the development of HIV-1 protease inhibitors that prevent the cleavage of viral polyproteins by obstructing the active site.

A fingerprint-based approach was employed with profile scaling as a virtual screening tool. The screening library consisted of 895 active molecules extracted from the MDL Data Drug Report (http://www.mdl.com/) (MDDR, 2007.2 version 2.3 SP2) and 123,331 background molecules present in the Asinex Platinum Collection of lead-like compounds (http://www.asinex.com/) (September 2007).

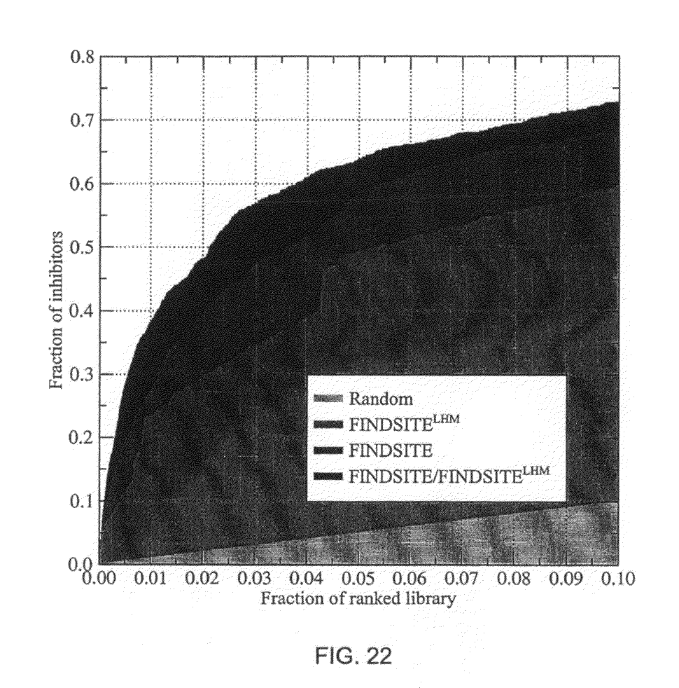

The results of HIV-1 protease virtual screening using ligand molecules predicted from homologous as well as weakly homologous threading templates were compared to the results obtained using known HIV-1 protease inhibitors. The performance on HIV inhibitors is presented in FIG. 22. A simple scoring function that considers only the coverage of the anchor portions of ligands bound to weakly related proteins (<35% sequence identity) selected by threading, especially when combined with a fingerprint-based approach, provided satisfactory enrichment in virtual screening. In fact, the molecular fingerprints constructed by FINDSITE (Brylinski, M. and J. Skolnick, A threading-based method (FINDSITE) for ligand-binding site prediction and functional annotation. Proc Natl Acad Sci USA, 2008. 105(1): p. 129-34) recovered slightly more known active compounds in the top-ranked fraction of the screening library than anchor-based FINDSITE^{LHM}; the enrichment factor calculated for the top 1% (10%) was 27.0 (6.8) and 23.3 (5.9) for FINDSITE and FINDSITE^{LHM}, respectively. Clearly, fusion by ranks outperforms the individual scoring functions with the enrichment factor of 38.1 (7.3) for the top 1% (10%) of ranked ligands. These results suggest that the anchor-based approach is able to detect active compounds for which the fingerprint-based method assigns relatively low score.

The foregoing description was intended to illustrate various aspects of the technology. It is not intended that the examples presented herein limit the scope of the appended claims. The invention now being fully described, it will be apparent to one of ordinary skill in the art that many changes and modifications can be made thereto without departing from the spirit or scope of the appended claims.

1. A method for identifying a binding site of a target protein, the method comprising:
obtaining a set of protein structure templates for the target protein;
selecting a subset of the protein structure templates such that, for each template in the subset, at least one bound ligand conformation is available;
optimizing a structural alignment of one or more templates in the subset to a structure of the target protein;
calculating a center of mass of each bound ligand conformation in the optimized structural alignment;
clustering the centers of mass of the bound ligand conformations in the optimized structural alignment, thereby identifying locations of one or more binding sites of the target protein.

2. A method of ranking two or more binding sites of a target protein, the method comprising:
identifying locations of the two or more binding sites of the target protein by the method of claim 1; and
assigning a rank to each of the two or more binding sites according to a number of bound ligand conformations clustered together at the location of each binding site.

3. A method of annotating a target protein with at least one molecular function, the method comprising:
ranking two or more binding sites of the target protein according to the method of claim 2;
for the highest ranked binding site:
retrieving, from a gene ontology database, gene ontology terms for one or more of the templates whose bound ligand conformations are clustered together in the binding site; and
annotating the target protein with the gene ontology terms.

4. A method of screening a target protein for ligands that bind to a binding site on the target protein, the method comprising:
identifying a location of the binding site of the target protein by the method of claim 1;
clustering the ligands in the optimized structural alignment of the bound ligand conformations for the binding site;
determining a set of equivalent atoms across the bound ligand conformations belonging to a cluster;
projecting the set of equivalent atoms into one or more functional groups;
constructing a representative ligand from the functional groups; and
comparing the representative ligand with a database of ligands.

5. The method of claim 4, further comprising:
predicting residues in the target protein that are involved in ligand binding;
docking compounds from the database of ligands into the binding site; and
identifying compounds with a high binding affinity to the target protein.

6. A method of treating an individual suffering from a disease mediated by a target protein, the method comprising:
administering to the individual a ligand identified as binding to the target protein by the method of claim 5, in an amount sufficient to produce a therapeutic effect.

7. The method of claim 1, wherein the obtaining a set of protein structure templates comprises:
threading a sequence of the target protein through a structure in a library of previously-determined protein structures, thereby obtaining an alignment score between the target protein and the structure; and
identifying the structure as a protein structure template for the target protein if the alignment score exceeds a threshold.

8. The method of claim 1, wherein the obtaining a set of protein structure templates comprises:
deducing an alignment of a sequence of the target protein to a sequence of a structure in a library of previously-determined protein structures, thereby obtaining a homology score between the target protein and the structure; and
identifying the structure as a protein structure template for the target protein if the similarity score exceeds a threshold.

9. The method of claim 1, wherein the set of protein structure templates are weakly homologous to the target protein.

10. The method of claim 1, wherein the protein structure templates comprise proteins from two or more families.

11. The method of claim 1, wherein the set of protein structure templates consists of 80-120 templates.

12. The method of claim 1, wherein the at least one bound ligand for which a conformation is available is selected from the group consisting of: organic molecules; cofactors; nucleotides; and peptides.

13. The method of claim 1, wherein the at least one bound ligand for which a conformation is available has from 6 to 200 atoms, not including hydrogen atoms.

14. The method of claim 1, wherein the at least one bound ligand for which a conformation is available makes a binding contact with at least 6 residues in the protein of the protein structure template.

15. The method of claim 1, wherein one or more of the protein structure templates are experimentally determined structures.

16. The method of claim 1, wherein one or more of the protein structure templates is a low-resolution structure.

17. The method of claim 1, wherein centers of mass of the ligands are clustered together using an 8 Å cut-off.

18. A method of for identifying a binding site of a target protein of known sequence, but whose experimental structure is not known or is known only to low resolution, the method comprising:
threading the sequence of the target protein through a set of protein structure templates that are only weakly homologous to the target protein;
selecting a subset of the protein structure templates that have a high threading score and are such that, for each template in the subset, at least one bound ligand conformation is available;
aligning the bound ligand conformations to a predicted or experimental structure of the target protein; and
associating aligned ligand conformations with a binding site of the target protein.

19. A method for identifying ligands that bind to a binding site of a target protein of known sequence, but whose experimental structure is not known or is known only to low resolution, the method comprising:
identifying the binding site using the method of claim 18;
using representative bound ligand conformations to search a database of ligands; and
selecting those ligands in the database of ligands that are predicted to have a high affinity of binding to the target protein.

20. The method of claim 1, further comprising presenting a result, and/or an intermediate stage, to a user.

21. A computer-readable medium, on which are stored executable instructions that, when executed by a computer processor, perform the method of claim 1.

22. A system, comprising:
a processor, configured to execute instructions;
a memory, on which are stored executable instructions, wherein the instructions are configured to perform the method of claim 1.
